(19) United States
(12) Patent Application Publication
(10) Pub. No.: US 2025/0281402 A1
(43) Pub. Date: Sep. 11, 2025

(54) COMPOSITIONS FOR LOCAL THERAPY DELIVERY TO BRAIN TUMORS AND METHODS

(71) Applicants: Massachusetts Institute of Technology, Cambridge, MA (US); The Brigham and Women's Hospital, Inc., Boston, MA (US)

(72) Inventors: Natalie Artzi, Brookline, MA (US); Michelle Zarrella Dion, Arlington, MA (US); Daniel Dahis, Cambridge, MA (US)

(21) Appl. No.: 18/859,682

(22) PCT Filed: Apr. 25, 2023
(86) PCT No.: PCT/US2023/019773

§ 371 Date: Oct. 24, 2024

Related U.S. Application Data
(60) Provisional application No. 63/334,643, filed on Apr. 25, 2022.

(51) Int. Cl. A61K 9/06; A61K 31/704; A61K 47/34; A61K 47/36; A61P 35/00

(52) U.S. Cl. CPC A61K 9/06; A61K 31/704; A61K 47/34; A61K 47/36; A61P 35/00

(57) ABSTRACT

Compositions for local delivery of a drug to an intracranial region, such as brain tissue or a brain tumor. Methods for locally delivering drug or therapy to an intracranial region. Compositions may include a hydrogel in which a chemotherapy drug or immunotherapy drug is dispersed. Kits that include compositions or solutions that may be combined to form compositions.

### CROSS-REFERENCE TO RELATED APPLICATIONS

This application claims priority to U.S. Provisional Patent Application No. 63/334,643, filed Apr. 25, 2022, which is incorporated by reference herein.

### BACKGROUND

Current therapies for brain cancer patients, such as glioblastoma (GBM) patients, are limited for various reasons, which has typically resulted in dismal patient prognoses. From 1971 to 2011, survival rates for brain cancer improved from about 8% to about 13%, whereas average survival rates from all cancers increased from about 25% to 50% during the same period.

Glioblastoma is the most common and aggressive primary central nervous system tumor, compromising about 48% of all malignant brain tumors, with the median age at diagnosis being 65 years of age. Standard treatment for GBM includes surgical resection, followed by concomitant chemotherapy and radiation. Surgery provides clinical relief, allows for diagnostic tissue samples to be collected, and improves survival. However, it is impossible to remove all cells as GBM has a diffuse infiltrative pattern and resection must be balanced with the preservation of healthy tissue. Radiotherapy and chemotherapy, as a result, are used to eliminate residual tumor cells following surgery, however, these techniques are limited by their inability to reach all tumor cells, their lack of specificity for tumor cells, and the ability of tumor cells to develop resistance to these therapies.

About 95% of patients are diagnosed after 40 years of age (median age=65 years). Primary GBM tumors arise de novo and account for 90% of cases, whereas secondary tumors arise from lower-grade gliomas and account for 10% of all tumors. Patients with both primary and secondary tumors typically present symptoms of increased intracranial pressure, such as headaches, neurological defects, seizures, etc. The diagnosis of GBM is based on the presence of several histological features, including anaplasia, mitotic activity, microvascular proliferation and necrosis

Current GBM therapy typically includes surgical resection, radiation therapy, and oral temozolomide chemotherapy, and this therapy regimen has resulted in a median overall survival time of about 15 months.

The ability to achieve more efficacious therapy has been limited by a number of factors, such as drug toxicity, drug delivery, and tumor biology. The toxicity of certain drugs can cause hypohysitis, uveitis and orbital inflammation, pneumonitis, adrenal insufficiency, enterocolitis, arthralgia, pacreatitis and auto-immune diabetes, rash and vitiligo, hepatitis, hypothyroidism, dry mouth, or a combination thereof.

It is believed that the GLIADEL® wafer is the only FDA-approved product that directly addresses challenges related to therapeutic delivery to the brain, but there are several drawbacks with this product, including wafer migration, a mechanical mismatch with soft tissue, rapid drug release, slow material degradation, and/or its use of less potent chemotherapies, which may be susceptible to resistance. For comparison purposes, a plot of the survival percentages of 9L glioma-bearing rats when treated with doxorubicin-loaded polyanhdyride wafers (i.e., the GLIADEL® delivery technology) is provided at FIG. 1.

There remains a need for improved compositions and methods for treating brain cancers, such as GBM, including compositions and methods for the local delivery of various therapies to brain tumors that overcome one or more of the foregoing disadvantages.

### BRIEF SUMMARY

Provided herein are methods and compositions, such as hydrogels (e.g., adhesive hydrogels), that may effectively deliver therapeutics to brain tumors or brain tissues. In some embodiments, the compositions include spray dispersion drug-loaded hydrogels. The compositions described herein may be injected during biopsy or via an intraventricular shunt. The compositions and methods described herein may achieve increased drug diffusion from resection cavities to disseminate tumor foci, provide alternative therapies to temozolomide, have a safe product profile following treatment with radiation therapy, facilitate wound healing, and/or reduce or eliminate scar formation.

Embodiments of the compositions provided herein include hydrogels, which may be soft and/or adhesive, and may provide competitive advantages over other products, such as the GLIADEL® wafer (Azurity Pharmaceuticals, USA). For example, the compositions provided herein may provide higher hydrogel: tissue interface, improved stability at resection site due to adhesiveness, a mechanical stiffness equivalent to soft tissue, slower and/or more controlled drug delivery than wafers, and/or the potential for combination delivery. The compositions provided herein also may delivery doxorubicin, which may be more potent and less susceptible to resistance than current therapies, and/or have multiple mechanisms of action, including the ability to induce immunogenic cell death.

In one aspect, drug delivery compositions are provided. In some embodiments, the drug delivery compositions include a hydrogel, and a drug dispersed in the hydrogel. The drug may include a chemotherapy drug, an immunotherapy drug, or a combination thereof. In some embodiments, the hydrogel includes a polymer component, wherein the polymer component includes a polymer having three or more aldehyde groups; and a dendrimer component, wherein the dendrimer component includes a dendrimer having at least 2 branches with one or more surface groups. The one or more surface groups may react, reversibly or irreversibly, with the three or more aldehyde groups.

In another aspect, methods of treating a patient are provided. In some embodiments, the methods include locally delivering a drug delivery composition as described herein to an intracranial region of the patient. The locally delivering of the drug delivery composition may include injecting or spraying the drug delivery composition in the intracranial region, which may include brain tissue, a brain tumor, a site of a previously resected tumor, or a combination thereof.

In yet another aspect, kits are provided. In some embodiments, the kits include a first part which includes a first solution including a polymer component, wherein the polymer component comprises a polymer; and a second part which includes a second solution including a dendrimer component, wherein the dendrimer component includes a dendrimer having at least 2 branches with one or more surface groups; wherein at least one drug is disposed in the first solution, the second solution, or both the first solution and the second solution, and wherein the drug includes a chemotherapy drug, an immunotherapy drug, or a combination thereof.

Additional aspects will be set forth in part in the description which follows, and in part will be obvious from the description, or may be learned by practice of the aspects described herein. The advantages described herein may be realized and attained by means of the elements and combinations particularly pointed out in the appended claims. It is to be understood that both the foregoing general description and the following detailed description are exemplary and explanatory only and are not restrictive.

### BRIEF DESCRIPTION OF THE DRAWINGS

FIG. 1 depicts survival percentages of various treatments.

FIG. 2 depicts an embodiment of a multi-compartment syringe.

FIG. 3 depicts a plot of cell viability v. concentration for various treatments.

FIG. 4 depicts degradation rates of embodiments of hydrogels.

FIG. 5A, FIG. 5B, FIG. 6A, FIG. 6B, FIG. 7A, FIG. 7B, FIG. 8A, and FIG. 8B depict release profiles of embodiments of compositions.

FIG. 9 depicts the cumulative release of doxorubicin from embodiments of compositions.

FIG. 10 depicts the cumulative release of capped doxorubicin from an embodiment of a composition.

FIG. 11 depicts the cumulative release of doxorubicin and an embodiment of an encapsulated doxorubicin from an embodiment of a composition.

FIG. 12A and FIG. 12B depict drug release profiles from an embodiment of a composition having different loadings of drug.

FIG. 13 depicts a plot of average radiant efficiency for embodiments of compositions.

FIG. 14 depicts a plot of the probability of survival for embodiments of compositions.

FIG. 15 depicts a plot of the probability of survival for an embodiment of a composition and an embodiment of an empty hydrogel.

FIG. 16 depicts a plot of the probability of survival for embodiments of compositions.

FIG. 17 depicts a plot of total flux for embodiments of compositions.

FIG. 18 depicts a plot of total flux for embodiments of compositions.

FIG. 19 depicts a plot of survival percentage for embodiments of compositions.

FIG. 20A and FIG. 20B depict plots of cell viability for embodiments of compositions.

FIG. 21 depicts a plot of the probability of survival after the administration of an embodiment of a composition.

FIG. 22 depicts a plot of the probability of survival after the administration of an embodiment of a composition.

FIG. 23 depicts a plot of the probability of survival after the administration of an embodiment of a composition.

FIG. 24 depicts a plot of the probability of survival after the administration of an embodiment of a composition.

FIG. 25A depicts a plot of the probability of survival after the administration of an embodiment of a composition containing 7.5 micrograms of an embodiment of a drug.

FIG. 25B depicts a plot of the probability of survival after the administration of an embodiment of a composition containing 15 micrograms of an embodiment of a drug.

FIG. 25C depicts a plot of the probability of survival after the administration of an embodiment of a composition containing 7.5 micrograms of an embodiment of a drug.

FIG. 26 depicts a plot of survival percentages after the administering of embodiments of compositions.

FIG. 27 depicts a plot of the probabilities of survival after the administering of embodiments of compositions.

FIG. 28 depicts a plot of the probabilities of survival after the administering of embodiments of compositions.

FIG. 29 depicts a plot of total flux after injection of embodiments of hydrogels.

FIG. 30 depicts data collected from tests to determine the impact of embodiments of compositions on T cells in the lymph nodes.

FIG. 31 depicts data collected from tests to determine the impact of embodiments of compositions on central memory CD8 T cells in the spleen.

FIG. 32 depicts data collected from tests to determine whether macrophages contributed to increase in CD45+ cells in a tumor upon treatment with an embodiment of a composition.

FIG. 33 depicts the results of a test of M1 polarization marker in CD80+/Macs 206 tumors treated with an embodiment of a composition.

FIG. 34 depicts the results of a test configured to determine shifts in dendritic cell populations.

FIG. 35 is a schematic of an embodiment of anticancer immune activity.

FIG. 36 is a schematic of the likely behavior of nanoparticles in the cGAS-STING pathway.

FIG. 37 is a schematic of a possible mechanism of action.

FIG. 38 depicts an embodiment of a nanoparticle-based STING agonist formulation.

FIG. 39 depicts data demonstrating the stimulation of dendritic cells and macrophages by an embodiment of a cyclic dinucleotide nanoparticle.

FIG. 40A and FIG. 40B depict total release and cumulative release profiles, respectively, of embodiments of compositions.

FIG. 41 depicts the results of a local immunotherapy pilot study.

FIG. 42 is a schematic of a treatment mechanism and results.

FIG. 43A and FIG. 43B depict results regarding cell viability and CRT expression, respectively, upon treatment with an embodiment of a composition.

FIG. 44 is a schematic of a study described herein.

FIG. 45 depicts the results of a study of BMDM CD86 activation marker expression.

FIG. 46 depicts results indicating that combination therapy increased the ratio of pro-inflammatory to anti-inflammatory BMDMs.

FIG. 47 is a schematic of an embodiment of treatment plan.

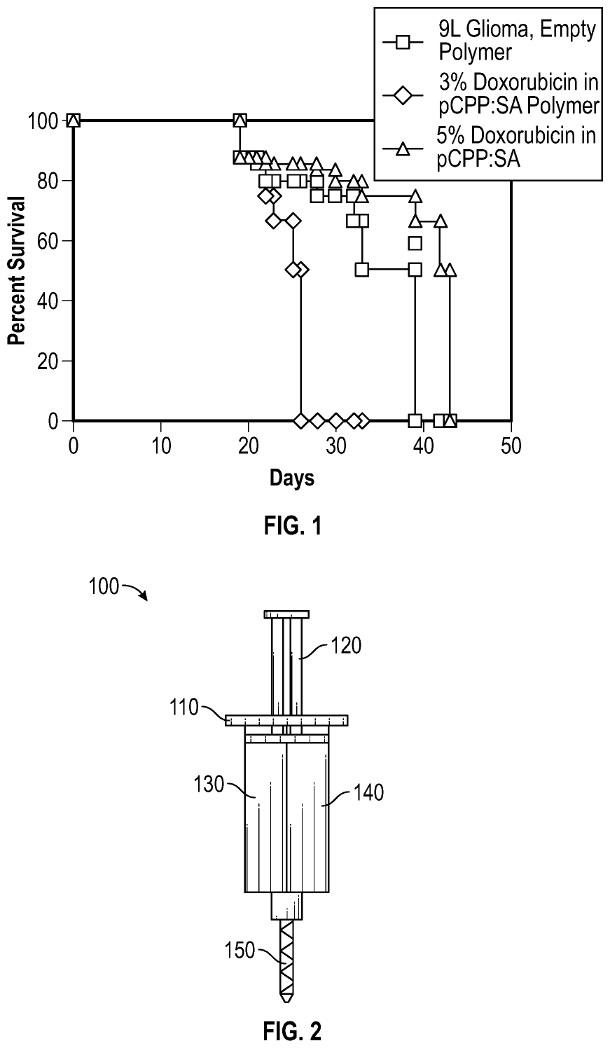

### DETAILED DESCRIPTION

The drug delivery compositions described herein may include a hydrogel and a drug. The drug may be selected from a chemotherapy drug, an immunotherapy drug, or a combination thereof. In some embodiments, the drug includes a nucleic acid, nanoparticles, antibodies, small molecules, immune modulating agents, or a combination thereof. The hydrogels may include those known in the art. Non-limiting examples of hydrogels are described, for example, in U.S. Pat. Nos. 8,802,072, and 10,736,914, which are incorporated by reference herein.

The drug delivery compositions described herein may include a hydrogel, and a drug dispersed in the hydrogel. The drug may be substantially evenly dispersed in the hydrogel.

In some embodiments, a drug is dispersed in the hydrogel in a manner that creates a concentration gradient of the drug.

The hydrogels, as described herein, may include a polymer component, wherein the polymer component includes a polymer having three or more aldehyde groups; and a dendrimer component, wherein the dendrimer component includes a dendrimer having at least 2 branches with one or more surface groups.

### Drug

The drugs of the drug delivery compositions described herein may include a chemotherapy drug, an immunotherapy drug, or a combination thereof.

The chemotherapy drug may include any known chemotherapy drug, including, but not limited to, bleomycin, busulfan, carboplatin, carmustine, cisplatin, cladbrbine, dactinomycin, daunorubicin, doxorubicin, estramustine, interferon, irinotecan, levamisole, methotrexate, mitomycin, paclitaxel, pentostatin, plicamycin, tamoxifen, temozolomide, vinblastine, vindesine, and the like. Additionally or alternatively, the chemotherapy drug may include one or more radiosensitizers including 5-halo-uracils, anti-angiogenesis compounds including thalidomide and tranilast, natural or synthetic peptide hormones including octreotide, and compounds that induce apoptosis including butyrate. In some embodiments, the chemotherapy drug includes doxorubicin.

The immunotherapy drug may include any known immunotherapy drug. As used herein, the phrase immunotherapy drug refers to any drug that is capable of inducing, enhancing, suppressing, or otherwise modifying an immune response. In some embodiments, the immunotherapy drug is selected from those that target (a) CTLA-4, such as ipilimumab or tremelimumab; (b) PD-1, such as nivolumab, pidilizumab or pembrolizumab, AMP-224; (c) PD-L1, such as MPDL-3280A, MSB0010718C or MEDI4736; or (d) GITR such as TRX518 or MK4166. Non-limiting examples of immunotherapy drugs are disclosed by U.S. Pat. No. 11,186,640.

In some embodiments, the immunotherapy drug includes a cyclic dinucleotide. A “cyclic dinucleotide” or “CDN” may include a class of molecules including 2′-5′ and/or 3′-5′ phosphodiester linkages between two purine nucleotides. This includes 2′-5′-2′,5′, 2′-5′-3′5′, and 3′,5′-3′,5′ linkages. CDNs may activate the cytosolic surveillance pathway through direct binding of two cytosolic pattern recognition receptors (PRRs), DEAD (aspartate-glutamate-alanine-aspartate)-box helicase 41 (DDX41) and STimulator of Interferon Genes (STING).

The Type I interferon response to infection by intracellular bacteria may result from the secretion of cyclic di-adenosine mono phosphate (cdAMP) or its related cyclic dinucleotide (CDN), cyclic di-guanine mono phosphate (cdGMP). CDNs may bind with high affinity to DDX41, and complex with the STING adaptor protein, resulting in the activation of the TBK1/IRF3 signaling pathway, and induction of IFN-β and other IRF-3 dependent gene products that strongly activate innate immunity. CDNs may include second messengers expressed by most bacteria and regulate diverse processes, including motility and formation of biofilms. Endogenous CDNs also may be produced in response to cytosolic DNA by the host enzyme cyclic guanosine monophosphate-adenosine monophosphate synthase (cGAS) in tumors or during infection. In some embodiments, a CDN is the canonical bacterial CDN, cyclic di-guanine mono phosphate (cdGMP). In some embodiments, a CDN is the endogenous product of cGAS. In some embodiments, a CDN is an agonist of STING. Cyclic dinucleotides that may be used include those known in the art, such as those disclosed in U.S. Pat. Nos. 7,709,458 and 7,592,326; WO 2007/054279; U.S. Patent Application Publication No. 2014/0205653; and Yan et al. Bioorg. Med. Chem Lett. 18:5631 (2008). Non-limiting examples of cyclic dinucleotides include cdAMP, cdGMP, cdIMP, c-AMP-GMP, c-AMP-IMP, and c-GMP-IMP, and analogs thereof including, but not limited to, phosphorothioate analogues.

In some embodiments, a cyclic dinucleotide is an agonist of STING (STimulator of Interferon Genes). As described herein, the STING signaling pathway in immune cells may be a central mediator of innate immune response and when stimulated, may induce expression of various interferons, cytokines and T cell recruitment factors that amplify and strengthen immune activity. Recent work has shown that STING agonists may be effective adjuvants and efficiently elicit an immune response, described, for example in Dubensky, T., et al., Therapeutic Advances in Vaccines, Vol. 1 (4): 131-143 (2013); and Hanson, M., et al., The Journal of Clinical Investigation, Vol. 125 (6): 2532-2546 (2015). In some embodiments, a STING agonist is chemically synthesized. In some embodiments, a STING agonist is an analog of a naturally occurring cyclic dinucleotide. STING agonists, including analogs of cyclic dinucleotides, suitable for use in the disclosure are provided in U.S. Pat. Nos. 7,709,458 and 7,592,326; and U.S. Patent Application Publication No. 2014/0205653.

A drug, such as a chemotherapy drug and/or immunotherapy drug (e.g., a cyclic dinucleotide) may be encapsulated in a liquid. The encapsulation may occur before the drug is dispersed in a hydrogel. For example, a chemotherapy drug may be encapsulated in a liquid, an immunotherapy drug (e.g., a cyclic dinucleotide) may be encapsulated in a liquid, or a chemotherapy drug and an immunotherapy drug (e.g., cyclic dinucleotide) may be encapsulated together in the same liquid or separately in different liquids. The encapsulated drugs may be in the form of liquid nanoparticles (e.g., nanospheres), such as any liquid nanoparticle known in the art. Non-limiting examples of encapsulating liquid nanoparticles are disclosed by U.S. Pat. No. 11,207,418, which is incorporated by reference. In some embodiments, the chemotherapy drug is doxorubicin, and the doxorubicin may be encapsulated in a liquid particle (e.g., a liquid sphere). The encapsulated doxorubicin may be DOXIL® anthracycline topoisomerase inhibitor (Baxter, USA). The nanoparticles may have any suitable average diameter, such as about 1 nm to about 100 nm, about 10 nm to about 90 nm, about 20 nm to about 80 nm, about 30 nm to about 70 nm, or about 40 nm to about 60 nm.

The drug described herein may be present in the drug delivery compositions at any amount. In some embodiments, drug is present in the drug delivery composition at a total amount of about 1 μg to about 5000 μg, about 1 μg to about 1000 μg, about 1 μg to about 500 μg, about 50 μg to about 500 μg, or about 50 μg to about 500 μg. For example, a composition that includes a “total amount” of 500 μg of drug may include 500 μg of a chemotherapy drug; 500 μg of an immunotherapy drug; 400 μg of a chemotherapy drug and 100 μg of an immunotherapy drug; etc. The drug described herein may be present in the drug delivery composition at any concentration. In some embodiments, drug is present in the drug delivery composition at a total concentration of about 0.1 μg/μL to about 100 μg/μL, about 1 μg/μL to about 80 μg/μL, about 1 μg/μL to about 60 μg/μL, about 1 μg/μL to about 40 μg/μL, about 1 μg/μL to about 30 μg/μL, about 1 μg/μL to about 20 μg/μL, about 1 μg/μL to about 10 μg/μL, about 2 μg/μL to about 8 μg/μL, or about 2 μg/μL to about 5 μg/μL.

### Methods of Treatment

Methods of treating patients are provided herein, including patients having a brain tumor. The patient may be any mammal, such as a human. In some embodiments, the methods of treating a patient include locally delivering a drug delivery composition as described herein to an intracranial region of the patient.

The local delivery of the drug delivery composition may be achieved in any manner. In some embodiments, the locally delivering of the drug delivery composition includes injecting or spraying the drug delivery composition. The injecting of the drug delivery composition may occur during biopsy, via an intraventricular shunt, or a combination thereof.

The intracranial region may include brain tissue, a brain tumor, a site of a previously resected tumor, or a combination thereof.

In some embodiments, the methods also include administering a second drug or therapy to the patient before, during, and/or after the locally delivering of the drug delivery composition. The administering of the second drug or therapy may be achieved in any manner, such as orally administering the second drug or therapy. The second drug may include a second chemotherapy drug or therapy, such as temozolomide or a PDI checkpoint blockade therapy.

The hydrogels described herein may provide for controlled drug release, such as sustained drug release. The components of the hydrogels may be tailored to achieve a desired release profile. The drug may be released continuously or intermittently. In some embodiments, the drug is released from the drug delivery composition continuously for at least 12 hours, at least 24 hours, at least 36 hours, at least 48 hours, at least 72 hours, at least 100 hours, at least 200 hours, or at least 300 hours.

In some embodiments, a cumulative percentage of the drug released from the drug delivery composition is at least 80%, 85%, or 90%, by weight, of the drug within 10 days or less, 12 days or less, 14 days or less, 16 days or less, 18 days or less, 20 days or less, 22 days or less, 24 days or less, 26 days or less, 28 days or less, 30 days or less, 32 days or less, 34 days or less, 36 days or less, 38 days or less, 40 days or less, 50 days or less, 60 days or less, 80 days or less, or 100 days or less after the locally delivering of the drug delivery composition.

### Dendrimer Component

In some embodiments, the dendrimer component comprises a dendrimer having amines on at least a portion of its surface groups, which are commonly referred to as “terminal groups” or “end groups.” The dendrimer may have amines on from 20% to 100% of its surface groups. In some embodiments, the dendrimer has amines on 100% of its surface groups. In some embodiments, the dendrimer component includes a dendrimer having amines on less than 75% of its surface groups. As used herein, the term “dendrimer” refers to any compound with a polyvalent core covalently bonded to two or more dendritic branches. In some embodiments, the polyvalent core is covalently bonded to three or more dendritic branches. In some embodiments, the amines are primary amines. In some embodiments, the amines are secondary amines. In some embodiments, one or more surface groups have at least one primary and at least one secondary amine.

In some embodiments, the dendrimer extends through at least 2 generations. In some embodiments, the dendrimer extends through at least 3 generations. In some embodiments, the dendrimer extends through at least 4 generations. In some embodiments, the dendrimer extends through at least 5 generations. In some embodiments, the dendrimer extends through at least 6 generations. In some embodiments, the dendrimer extends through at least 7 generations.

In some embodiments, the dendrimer has a molecular weight of from about 1,000 to about 1,000,000 Daltons. In some embodiments, the dendrimer has a molecular weight of from about 3,000 to about 120,000 Daltons. In some embodiments, the dendrimer has a molecular weight of from about 10,000 to about 100,000 Daltons. In some embodiments, the dendrimer has a molecular weight of from about 20,000 to about 40,000 Daltons. Unless specified otherwise, the “molecular weight” of the dendrimer refers to the weight average molecular weight.

Generally, the dendrimer may be made using any known methods. In some embodiments, the dendrimer is made by oxidizing a starting dendrimer having surface groups comprising at least one hydroxyl group so that at least a portion of the surface groups comprise at least one amine. In some embodiments, the dendrimer is made by oxidizing a starting generation 5 (G5) dendrimer having surface groups comprising at least one hydroxyl group so that at least a portion of the surface groups comprise at least one amine. In some embodiments, the dendrimer is made by oxidizing a starting G5 dendrimer having surface groups comprising at least one hydroxyl group so that about 25% of the surface groups comprise at least one amine. In some embodiments, the dendrimer is a G5 dendrimer having primary amines on about 25% of the dedrimer's surface groups.

In some embodiments, the dendrimer is a poly(amidoamine)—derived (PAMAM) dendrimer. In some embodiments, the dendrimer is a G5 PAMAM-derived dendrimer. In some embodiments, the dendrimer is a G5 PAMAM-derived dendrimer having primary amines on about 25% of the dendrimer's surface groups. In some embodiments, the dendrimer is a poly(propyleneimine)—derived dendrimer.

In some embodiments, the dendrimer component is combined with a liquid to form a dendrimer component solution. In some embodiments, the dendrimer component solution is an aqueous solution. In some embodiments, the solution comprises water, phosphate buffer saline (PBS), Dulbecco's Modified Eagle's Medium (DMEM), or any combination thereof. In some embodiments, the dendrimer component concentration in the dendrimer component solution is about 5% to about 25% by weight. In some embodiments, the dendrimer component concentration in the dendrimer component solution is about 10% to about 20% by weight. In some embodiments, the dendrimer component concentration in the dendrimer component solution is about 11% to about 15% by weight.

In some embodiments, the dendrimer component or dendrimer component solution further includes one or more additives. Generally, the amount of additive may vary depending on the application, tissue type, concentration of the dendrimer component solution, the type of dendrimer component, concentration of the polymer component solutions, and/or the type of polymer component. Example of suitable additives, include but are not limited to, pH modifiers, thickeners, antimicrobial agents, colorants, surfactants, and radio-opaque compounds. Specific examples of these types of additives are described herein. In one embodiment, the dendrimer component solution comprises a foaming additive.

### Polymer Component

Generally, the polymer component includes a polymer and/or oligomer with one or more functional groups capable of reacting with one or more functional groups on a biological tissue and/or one or more functional groups on the dendrimer component.

In some embodiments, the polymer is at least one polysaccharide. In some embodiments, the at least one polysaccharide may be linear, branched, or have both linear and branched sections within its structure. Generally, the at least one polysaccharide may be natural, synthetic, or modified—for example, by cross-linking, altering the polysaccharide's substituents, or both. In one embodiment, the at least one polysaccharide is plant-based. In some embodiments, the at least one polysaccharide is animal-based. In some embodiments, the at least one polysaccharide is a combination of plant-based and animal-based polysaccharides. Non-limiting examples of polysaccharides include, but are not limited to, dextran, chitin, starch, agar, cellulose, hyaluronic acid, or a combination thereof.

In some embodiments, the at least one polymer has a molecular weight of from about 1,000 to about 1,000,000 Daltons. In some embodiments, the at least one polymer has a molecular weight of from about 5,000 to about 15,000 Daltons. Unless specified otherwise, the “molecular weight” of the polymer refers to the weight average molecular weight.

In some embodiments, the polymer is functionalized so that its structure includes one or more functional groups that will react with one or more functional groups on a biological tissue and/or one or more functional groups on the dendrimer component. In some embodiments, the polymer is functionalized so that its structure includes three or more functional groups that will react with one or more functional groups on a biological tissue and/or one or more functional groups on the dendrimer component. In some embodiments, the functional groups incorporated into the polymer's structure is aldehyde.

In some embodiments, the polymer's degree of functionalization is adjustable. The “degree of functionalization” generally refers to the number or percentage of groups on the polymer that are replaced or converted to the desired one or more functional groups. The one or more functional groups, in particular embodiments, include aldehydes, substituents capable of photoreversible dimerization, or a combination thereof. In some embodiments, the degree of functionalization is adjusted based on the type of tissue to which the adhesive is applied, the concentration(s) of the components, and/or the type of polymer or dendrimer used in the adhesive. In some embodiments, the degree of functionalization is from about 10% to about 75%. In some embodiments, the degree of functionalization is from about 15% to about 50%. In some embodiments, the degree of functionalization is from about 20% to about 30%.

In some embodiments, the polymer is dextran with a molecular weight of about 10 kDa. In some embodiments, the polymer is dextran having about 50% of its hydroxyl group converted to aldehydes. In some embodiments, the polymer is dextran with a molecular weight of about 10 kDa and about 50% of its hydroxyl groups converted to aldehydes.

In some embodiments, a polysaccharide is oxidized to include a desired percentage of one or more aldehyde functional groups. A polysaccharide, for example, may be oxidized to convert about 10% to about 100%, about 10% to about 90%, about 20% to about 80%, about 30% to about 70%, about 40% to about 60%, or about 50% (mole %) of its hydroxyl groups to aldehydes. Generally, this oxidation may be conducted using any known means. For example, suitable oxidizing agents include, but are not limited to, periodates, hypochlorites, ozone, peroxides, hydroperoxides, persulfates, and percarbonates. In one embodiment, the oxidation is performed using sodium periodate. Typically, different amounts of oxidizing agents may be used to alter the degree of functionalization.

In some embodiments, the polymer component is combined with a liquid to form a polymer component solution. In some embodiments, the polymer component solution is an aqueous solution. In some embodiments, the solution comprises water, PBS, DMEM, or any combination thereof.

Generally, the polymer component solution may have any suitable concentration of polymer component. In some embodiments, the polymer component concentration in the polymer component solution is about 5% to about 40% by weight. In some embodiments, the polymer component concentration in the polymer component solution is about 5% to about 30% by weight. In some embodiments, the polymer component concentration in the polymer component solution is about 5% to about 25% by weight. Typically, the concentration may be tailored and/or adjusted based on the particular application, tissue type, and/or the type and concentration of dendrimer component used.

The polymer component or polymer component solution may also include one or more additives. In some embodiments, the additive is compatible with the polymer component. In some embodiments, the additive does not contain primary or secondary amines. Generally, the amount of additive varies depending on the application, tissue type, concentration of the polymer component solution, the type of polymer component and/or dendrimer component. Examples of suitable additives, include, but are not limited to, pH modifiers, thickeners, antimicrobial agents, colorants, surfactants, radio-opaque compounds, and the other additives described herein. In other embodiments, the polymer component solution comprises a foaming agent.

In some embodiments, the pH modifier is an acidic compound. Examples of acidic pH modifiers include, but are not limited to, carboxylic acids, inorganic acids, and sulfonic acids. In some embodiments, the pH modifier is a basic compound. Examples of basic pH modifiers include, but are not limited to, hydroxides, alkoxides, nitrogen-containing compounds other than primary and secondary amines, basic carbonates, and basic phosphates.

Generally, the thickener may be selected from any known viscosity-modifying compounds, including, but not limited to, polysaccharides and derivatives thereof, such as starch or hydroxyethyl cellulose.

Generally, the surfactant may be any compound that lowers the surface tension of water. In one embodiment, the surfactant is an ionic surfactant—for example, sodium lauryl sulfate. In another embodiment, the surfactant is a neutral surfactant. Examples of neutral surfactants include, but are not limited to, polyoxyethylene ethers, polyoxyethylene esters, and polyoxyethylene sorbitan.

In some embodiments, the radio-opaque compound is barium sulfate, gold particles, or a combination thereof.

### Hydrogels

Generally, the hydrogels described herein may be formed by combining the polymer component or polymer component solution, and the dendrimer component or dendrimer component solution in any manner. In some embodiments, the polymer component or polymer component solution, and the dendrimer component or dendrimer component solution are combined before locally delivering the drug delivery composition. In some embodiments, the polymer component or polymer component solution, and the dendrimer component or dendrimer component solution are combined, in any order, in an intracranial region. In some embodiments, the locally delivering of a composition includes locally delivering the polymer component or polymer component solution to an intracranial region, and locally delivering the dendrimer component or dendrimer component solution to the intracranial region, wherein the polymer component and dendrimer component contact each other in the intracranial region.

The hydrogels may be locally delivered using any suitable tool and methods. Double barrel syringes with rigid or flexible discharge tips, and optional extension tubes, needles, stents, catheters, and other devices known in the art are envisioned.

As used herein, the hydrogel is a “treatment” when it stops, reverses, or reduces the rate of tumor growth, improves the probability of survival, extend a patient's life, or a combination thereof.

After contacting one or more biological tissues in an intracranial region, the hydrogels may be allowed adequate time to cure or gel. When the hydrogel “cures” or “gels,” as those terms are used herein, it means that the reactive groups on the polymer component, dendrimer component, and one or more biological tissues have undergone one or more reactions. Not wishing to be bound by any particular theory, it is believed that the hydrogels described herein are effective because the polymer component reacts with both the dendrimer component and the surface of the biological tissues. In some embodiments, the polymer component's aldehyde functional groups react with one or more amines of (i) a drug, such as doxorubicin, (ii) the dendrimer component, and/or and (iii) one or more biological tissues to form imine bonds. The forming of imine bonds may be reversible, and the formation and hydrolysis of imine bonds may control, or contribute to, release kinetics of a drug from a hydrogel; therefore, the number/ratio of functional groups that participate in imine bond formation may be configured to achieve desired release kinetics. In some embodiments, it is believed that the amines on the dendrimer component react with a high percentage of the aldehydes on the polymer component, thereby reducing toxicity and increasing biocompatibility of the hydrogels. Typically, the time needed to cure or gel the hydrogels will vary based on a number of factors, including, but not limited to, the characteristics of the polymer component and/or dendrimer component, the concentrations of the polymer component solution and/or the dendrimer component solution, and the characteristics of the one or more biological tissues. In some embodiments, the hydrogel will cure sufficiently to provide desired bonding or sealing shortly after the components are combined. The gelation or cure time should provide that a mixture of the components can be delivered in fluid form to a target area before becoming too viscous or solidified and then once applied to the target area sets up rapidly thereafter. In one embodiment, the gelation or cure time is less than 120 seconds. In some embodiments, the gelation or cure time is between 3 and 60 seconds. In some embodiments, the gelation or cure time is between 5 and 30 seconds. Before or after the hydrogel has cured, the substituents capable of photoreversible dimerization may be activated or deactivated as desired.

In certain embodiments, one or more foaming agents are added to the polymer component solution and/or the dendrimer component solution before the solutions are combined. In one embodiment, the foaming agents comprise a two part liquid system comprising Part 1 and Part 2, wherein Part 1 comprises a bicarbonate and Part 2 comprises an aqueous solution of di- or polyaldehydes and a titrant. A wide range of di- or polyaldehydes exist, and their usefulness is restricted largely by availability and by their solubility in water. For example, aqueous glyoxal (ethanedial) is useful, as is aqueous glutaraldehyde (pentadial). Water soluble mixtures of di- and polyaldehydes prepared by oxidative cleavage of appropriate carbohydrates with periodate, ozone or the like may also be useful.

A titrant is most preferably employed in the liquid solution of Part 2. More specifically, the titrant is an organic or inorganic acid, buffer, salt, or salt solution which is capable of reacting with the bicarbonate component of Part 1 to generate carbon dioxide and water as reaction by-products. The carbon dioxide gas that is generated creates a foam-like structure of the hydrogel and also causes the volume of the hydrogel to expand.

Most preferably, the titrant is an inorganic or organic acid that is present in an amount to impart an acidic pH to the resulting mixture of the Part 1 and Part 2 components. Preferred acids that may be employed in the practice of the present invention include phosphoric acid, sulfuric acid, hydrochloric acid, acetic acid, and citric acid.

### Hydrogel Kits

In another aspect, a kit is provided that comprises a first part that includes a polymer component or polymer component solution, and a second part that includes a dendrimer component or dendrimer component solution. The kit may further include an applicator or other device means, such as a multi-compartment syringe, for storing, combining, and delivering the two parts and/or the resulting hydrogel to an intracranial region.

At least one drug, as described herein, may be disposed in the polymer component solution, the dendrimer component solution, or both the polymer component solution and the dendrimer component solution. The drug, as described herein, may include a chemotherapy drug, an immunotherapy drug, or a combination thereof.

In some embodiments, the kit comprises separate reservoirs for the polymer component solution and the dendrimer component solution. In some embodiments, the kit comprises reservoirs for polymer component solutions of different concentrations. In some embodiments, the kit comprises reservoirs for dendrimer component solutions of different concentrations.

In some embodiments, the kit comprises instructions for selecting an appropriate concentration or amount of at least one of the polymer component, polymer component solution, dendrimer component, dendrimer component solution, or drug to compensate or account for at least one characteristic of one or more biological tissues, treatment plans, etc.

In some embodiments, the kit comprises at least one syringe. In some embodiments, the syringe comprises separate reservoirs for the polymer component solution and the dendrimer component solution. The syringe may also comprise a mixing tip that combines the two solutions as the plunger is depressed. The mixing tip may be releasably securable to the syringe (to enable exchange of mixing tips), and the mixing tip may comprise a static mixer. In some embodiments, the reservoirs in the syringe may have different sizes or accommodate different volumes of solution. In some embodiments, the reservoirs in the syringe may be the same size or accommodate the same volumes of the solution. In some embodiments, one reservoir may comprise Part 1 of the foaming composition described hereinabove, and a second reservoir may comprise Part 2 of the foaming composition.

FIG. 2 depicts one embodiment of a syringe 100. The syringe 100 includes a body 110 with two reservoirs (130, 140). A dendrimer component solution is disposed in the first reservoir 130, and a polymer component solution is disposed in the second reservoir 140. The two reservoirs (130, 140) are emptied by depressing the plunger 120, which pushes the contents of the two reservoirs (130, 140) into the mixing tip 150 and out of the syringe 100. In a further embodiment, one or more of the reservoirs of the syringe may be removable. In this embodiment, the removable reservoir may be replaced with a reservoir containing a polymer component solution or a dendrimer component solution of a desired concentration. In a preferred embodiment, the kit is sterile. For example, the components of the kit may be packaged together, for example in a tray, pouch, and/or box. The packaged kit may be sterilized using known techniques at suitable wavelengths (where applicable), such as electron beam irradiation, gamma irradiation, ethylene oxide sterilization, or other suitable techniques.

All referenced publications are incorporated herein by reference in their entirety. Furthermore, where a definition or use of a term in a reference, which is incorporated by reference herein, is inconsistent or contrary to the definition of that term provided herein, the definition of that term provided herein applies and the definition of that term in the reference does not apply.

While certain aspects of conventional technologies have been discussed to facilitate disclosure of various embodiments, applicants in no way disclaim these technical aspects, and it is contemplated that the present disclosure may encompass one or more of the conventional technical aspects discussed herein.

The present disclosure may address one or more of the problems and deficiencies of known methods and processes. However, it is contemplated that various embodiments may prove useful in addressing other problems and deficiencies in a number of technical areas. Therefore, the present disclosure should not necessarily be construed as limited to addressing any of the particular problems or deficiencies discussed herein.

In this specification, where a document, act or item of knowledge is referred to or discussed, this reference or discussion is not an admission that the document, act or item of knowledge or any combination thereof was at the priority date, publicly available, known to the public, part of common general knowledge, or otherwise constitutes prior art under the applicable statutory provisions; or is known to be relevant to an attempt to solve any problem with which this specification is concerned.

In the descriptions provided herein, the terms “includes,” “is,” “containing,” “having,” and “comprises” are used in an open-ended fashion, and thus should be interpreted to mean “including, but not limited to.” When compositions, kits, or methods are claimed or described in terms of “comprising” various steps or components, the compositions, kits, or methods can also “consist essentially of” or “consist of” the various steps or components, unless stated otherwise.

The terms “a,” “an,” and “the” are intended to include plural alternatives, e.g., at least one. For instance, the disclosure of “a polymer”, “a drug”, and the like, is meant to encompass one, or mixtures or combinations of more than one polymer, drug, and the like, unless otherwise specified.

Various numerical ranges may be disclosed herein. When Applicant discloses or claims a range of any type, Applicant's intent is to disclose or claim individually each possible number that such a range could reasonably encompass, including end points of the range as well as any sub-ranges and combinations of sub-ranges encompassed therein, unless otherwise specified. Moreover, all numerical end points of ranges disclosed herein are approximate. As a representative example, Applicant discloses, in some embodiments, that the drug is present in the drug delivery composition at a total concentration of about 1 μg/μL to about 10 μg/μL. This range should be interpreted as encompassing about 1 μg/μL and about 10 μg/μL, and further encompasses “about” each of 2 μg/μL, 3 μg/μL, 4 μg/μL, 5 μg/μL, 6 μg/μL, 7 μg/μL, 8 μg/μL, and 9 μg/μL, including any ranges and sub-ranges between any of these values.

As used herein, the term “about” means plus or minus 10% of the numerical value of the number with which it is being used.

### Listing of Embodiments

The following is a non-limiting listing of embodiments of the disclosure:
Embodiment 1. A drug delivery composition comprising a hydrogel, such as an adhesive hydrogel, and a drug dispersed in the hydrogel, wherein the drug comprises a chemotherapy drug, an immunotherapy drug, or a combination thereof.
Embodiment 2. The composition of Embodiment 1, wherein the hydrogel comprises a polymer component, such as a polymer component comprising a polymer having three or more aldehyde groups.
Embodiment 3. The composition of any of the preceding embodiments, wherein the hydrogel comprises a dendrimer component, such as a dendrimer component comprising a dendrimer having at least 2 branches with one or more surface groups.
Embodiment 4. The composition of any of the preceding embodiments, wherein 100% of the one or more surface groups comprise at least one primary or secondary amine.
Embodiment 5. The composition of any of the preceding embodiments, wherein less than 95%, less than 90%, less than 85%, less than 80%, or less than 75% of the one or more surface groups comprise at least one primary or secondary amine.
Embodiment 6. The composition of any of the preceding embodiments, wherein the dendrimer is a generation 5 polyamidoamine (G5 PAMAM) dendrimer.
Embodiment 7. The composition of any of the preceding embodiments, wherein the polymer having three or more aldehyde groups comprises a polysaccharide, such as an oxidized polysaccharide.
Embodiment 8. The composition of any of the preceding embodiments, wherein about 10% to about 100%, about 10% to about 90%, about 20% to about 80%, about 30% to about 70%, about 40% to about 60%, or about 50% (mole %) of the polysaccharide's hydroxyl groups are oxidized to aldehydes.
Embodiment 9. The composition of any of the preceding embodiments, wherein the polymer comprises dextran.
Embodiment 10. The composition of any of the preceding embodiments, wherein the drug comprises or consists of the chemotherapy drug.
Embodiment 11. The composition of any of the preceding embodiments, wherein the chemotherapy drug comprises bleomycin, busulfan, carboplatin, carmustine, cisplatin, cladbrbine, dactinomycin, daunorubicin, doxorubicin, estramustine, interferon, irinotecan, levamisole, methotrexate, mitomycin, paclitaxel, pentostatin, plicamycin, tamoxifen, temozolomide, vinblastine, vindesine, or a combination thereof.
Embodiment 12. The composition of any of the preceding embodiments, wherein the chemotherapy drug comprises one or more radiosensitizers, such as a 5-halo-uracil, an anti-angiogenesis compound, (such as thalidomide, tranilast, natural or synthetic peptide hormones, including octreotide, and/or compounds that induce apoptosis, including butyrate).
Embodiment 13. The composition of any of the preceding embodiments, wherein the chemotherapy drug comprises or consists of doxorubicin.
Embodiment 14. The composition of any of the preceding embodiments, wherein the immunotherapy drug comprises one or more agents that target (a) CTLA-4, such as ipilimumab or tremelimumab; (b) PD-1, such as nivolumab, pidilizumab or pembrolizumab, AMP-224; (c) PD-L1, such as MPDL-3280A, MSB0010718C or MEDI4736; (d) GITR, such as TRX518 or MK4166; or (e) a combination thereof.
Embodiment 15. The composition of any of the preceding embodiments, wherein the immunotherapy drug comprises or consists of a cyclic dinucleotide.
Embodiment 16. The composition of any of the preceding embodiments, wherein the chemotherapy drug, the immunotherapy drug, or the chemotherapy drug and the immunotherapy drug are encapsulated in a liquid, wherein the encapsulation may form a liquid particle (e.g., a liquid nanoparticle), such as a liquid sphere (e.g., a liquid nanosphere).
Embodiment 17. The composition of any of the preceding embodiments, wherein the chemotherapy drug and the immunotherapy drug are encapsulated together in the same liquid particle, or the chemotherapy drug and the immunotherapy drug are encapsulated separately in different liquid particles.
Embodiment 18. The composition of any of the preceding embodiments, wherein the liquid particle, such as a liquid nanoparticle, comprises a poly-beta-amino-ester.
Embodiment 19. The composition of Embodiment 18, wherein a cyclic dinucleotide is conjugated to the poly-beta-amino-ester via a cathepsin-sensitive bond, and wherein the poly-beta-amino-ester is optionally modified with arginine.
Embodiment 20. The composition of any of the preceding embodiments, wherein the liquid particle is a liquid nanoparticle (e.g., a liquid nanosphere) having an average diameter of about 1 nm to about 100 nm, about 10 nm to about 90 nm, about 20 nm to about 80 nm, about 30 nm to about 70 nm, or about 40 nm to about 60 nm.
Embodiment 21. The composition of any of the preceding embodiments, wherein the hydrogel comprises phosphate buffered saline (PBS).
Embodiment 22. The composition of any of the preceding embodiments, wherein the hydrogel has a solid content of about 5% to about 40%, about 5% to about 35%, about 5% to about 30%, about 5% to about 25%, about 8% to about 25%, about 10% to about 20%, or about 12% to about 18%, by weight.
Embodiment 23. The composition of any of the preceding embodiments, wherein the drug is present in the drug delivery composition at a total amount of about 1 μg to about 5000 μg, about 1 μg to about 1000 μg, about 1 μg to about 500 μg, about 50 μg to about 500 μg, or about 50 μg to about 500 μg.
Embodiment 24. The composition of any of the preceding embodiments, wherein the drug is present in the drug delivery composition at a total concentration of about 0.1 μg/μL to about 100 μg/μL, about 1 μg/μL to about 80 μg/μL, about 1 μg/μL to about 60 μg/μL, about 1 μg/μL to about 40 μg/μL, about 1 μg/μL to about 30 μg/μL, about 1 μg/μL to about 20 μg/μL, about 1 μg/μL to about 10 μg/μL, about 2 μg/μL to about 8 μg/μL, or about 2 μg/μL to about 5 μg/μL.
Embodiment 25. A method of treating a patient, the method comprising locally delivering the drug delivery composition of any of the preceding embodiments to the patient, such as to an intracranial region of the patient, wherein the patient, optionally, is a human.
Embodiment 26. The method of any of the preceding embodiments, wherein
   (A) the locally delivering of the drug delivery composition comprises injecting or spraying the drug delivery composition; and/or
   (B) the polymer component/solution and the dendrimer component/solution are contacted before the locally delivering of the composition to the intracranial region; and/or
   (C) the locally delivering of the drug delivery composition comprises locally delivering the polymer component/solution and locally delivering the dendrimer component/solution, in any order, sequentially, at least partially simultaneously, or a combination thereof to the intracranial region.
Embodiment 27. The method of any of the preceding embodiments, wherein the injecting of the drug delivery composition occurs during biopsy, via an intraventricular shunt, or a combination thereof.
Embodiment 28. The method of any of the preceding embodiments, wherein the intracranial region comprises brain tissue, a brain tumor, a site of a previously resected tumor, or a combination thereof.
Embodiment 29. The method of any of the preceding embodiments, further comprising administering a second drug or therapy to the patient before, during, and/or after the locally delivering of the drug delivery composition.
Embodiment 30. The method of any of the preceding embodiments, wherein the administering of the second drug or therapy comprises orally administering the second drug or therapy.
Embodiment 31. The method of any of the preceding embodiments, wherein the second drug or therapy comprises a second chemotherapy drug or therapy, including, but not limited to, any of those disclosed herein, such as any of those of Embodiment 11.
Embodiment 32. The method of any of the preceding embodiments, wherein the second chemotherapy drug or therapy is not present in the drug delivery composition.
Embodiment 33. The method of any of the preceding embodiments, wherein the second chemotherapy drug or therapy comprises or consists of temozolomide.
Embodiment 34. The method of any of the preceding embodiments, wherein the second drug or therapy comprises an immunotherapy drug, such as any of those disclosed herein, such as any of those of Embodiment 14 or 15.
Embodiment 35. The method of any of the preceding embodiments, wherein the second drug or therapy comprises aPD1 checkpoint blockade therapy.
Embodiment 36. The method of any of the preceding embodiments, wherein the drug is released from the drug delivery composition continuously for at least 12 hours, at least 24 hours, at least 36 hours, at least 48 hours, at least 72 hours, at least 100 hours, at least 200 hours, or at least 300 hours.
Embodiment 37. The method of any of the preceding embodiments, wherein a cumulative percentage of the drug released from the drug delivery composition is at least 80%, by weight, within 10 days or less, 12 days or less, 14 days or less, 16 days or less, 18 days or less, 20 days or less, 22 days or less, 24 days or less, 26 days or less, 28 days or less, 30 days or less, 32 days or less, 34 days or less, 36 days or less, 38 days or less, 40 days or less, 50 days or less, 60 days or less, 80 days or less, or 100 days or less after the locally delivering of the drug delivery composition.
Embodiment 38. The method of any of the preceding embodiments, wherein a cumulative percentage of the drug released from the drug delivery composition is at least 90%, by weight, within 10 days or less, 12 days or less, 14 days or less, 16 days or less, 18 days or less, 20 days or less, 22 days or less, 24 days or less, 26 days or less, 28 days or less, 30 days or less, 32 days or less, 34 days or less, 36 days or less, 38 days or less, 40 days or less, 50 days or less, 60 days or less, 80 days or less, or 100 days or less after the locally delivering of the drug delivery composition.
Embodiment 39. A kit for making a drug delivery composition, the kit comprising a first part which includes a first solution comprising the polymer component of any of the preceding embodiments; and a second part which includes a second solution comprising the dendrimer component of any of the preceding embodiments.
Embodiment 40. The kit of any of the preceding embodiments, wherein the at least one drug is disposed in the first solution, the second solution, or both the first solution and the second solution.
Embodiment 41. The kit of embodiment 40, wherein (i) the drug comprises or consists of a chemotherapy drug that is disposed in the first solution, the second solution, or both the first solution and the second solution, (ii) the drug comprises or consists of an immunotherapy drug that is disposed in the first solution, the second solution, or both the first solution and the second solution, or (iii) the drug comprises or consists of a chemotherapy drug and an immunotherapy drug that are both disposed in the first solution, both disposed in the second solution, both disposed in the first and the second solution, or the chemotherapy drug is disposed in one of the first and second solution, and the immunotherapy drug is disposed in the other.
Embodiment 42. The kit of any of the preceding embodiments, wherein the kit comprises a syringe.
Embodiment 43. The kit of any of the preceding embodiments, wherein the syringe comprises a mixing tip, such as the mixing tip depicted at FIG. 2.
Embodiment 44. The kit of any of the preceding embodiments, wherein the syringe comprises a first reservoir and a second reservoir, wherein the first solution and the second solution are disposed in the first reservoir and the second reservoir, respectively.
Embodiment 45. The composition, kit, or method of any of the preceding embodiments, wherein the aldehyde groups of the polymer component react, e.g., reversibly react, with one or more amines of (i) the drug, such as doxorubicin, (ii) the dendrimer component, and/or and (iii) one or more biological tissues to form imine bonds.
Embodiment 46. The composition, kit, or method of Embodiment 45, wherein the forming of the imine bonds and hydrolysis of the imine bonds controls, or contributes to, release kinetics of the drug from the hydrogel.
Embodiment 47. The composition, kit, or method of Embodiment 45, wherein the forming of the imine bonds and hydrolysis of the imine bonds achieves sustained delivery of drug.

### EXAMPLES

The present invention is further illustrated by the following examples, which are not to be construed in any way as imposing limitations upon the scope thereof. On the contrary, it is to be clearly understood that resort may be had to various other aspects, embodiments, modifications, and equivalents thereof which, after reading the description herein, may suggest themselves to one of ordinary skill in the art without departing from the spirit of the present invention or the scope of the appended claims. Thus, other aspects of this invention will be apparent to those skilled in the art from consideration of the specification and practice of the invention disclosed herein.

### Example 1-Testing of Various Therapies

The potencies of several therapies again GBM cells lines were measured. Doxorubicin demonstrated elevated potency again GBM cell lines compared to standard of care chemotherapies.

### Potency of Various Drugs

| Drug | IC₅₀ in GL261 (μM) | IC₅₀ in CT-2A (μM) |
|---|---|---|
| Temozolomide | >750 | >750 |
| Irinotecan | 106.8 | 150.1 |
| Carmustine (BCNU) | 145.9 | 155.8 |
| Acriflavine | 2.7 | 7.3 |
| Doxorubicin | 0.8 | 0.1 |

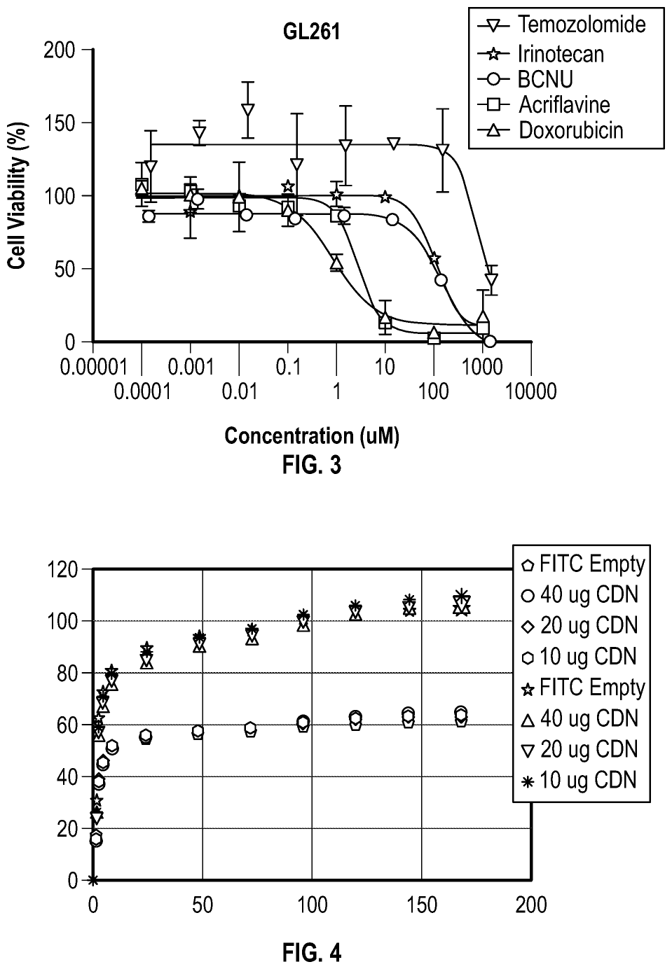

FIG. 3 depicts a plot of cell viability v. concentration of each of the therapies listed in the foregoing table.

The testing revealed that doxorubicin more potently eliminated GBM spheroids in vitro than other tested chemotherapies. The other tested chemotherapies, however, may be used in the methods and compositions described herein.

Based on these tests, however, an initial screening of dextran-dendrimer hydrogels was performed.

### Embodiments of Compositions

| | Antibody concentration (mg/mL) | | | | | | | |
|---|---|---|---|---|---|---|---|---|
| Formulation | | | 40 | | 80 | | 120 | |
| (prior to | | | (200 μg | | (400 μg | | (600 μg | |
| mixing) | 0 | | dose) | | dose) | | dose) | |
| Sample 1- 50% | 93 | seconds | 113 | seconds | 109 | seconds | >120 | seconds |
| oxidized dextran | 2+ | min | 2+ | min | 2+ | min | n.t. | |
| (12.5% w/v), | | | | | | | | |
| PAMAM dendrimer | | | | | | | | |
| (15% w/v), pH 8.8 | | | | | | | | |
| Sample 2- 75% | 53 | seconds | 54 | seconds | 70 | seconds | 101 | seconds |
| oxidized dextran | <1.5 | min | <1.5 | min | <1.5 | min | n.t. | |
| (12.5% w/v), | | | | | | | | |
| PAMAM dendrimer | | | | | | | | |
| (15% w/v), pH 8.8 | | | | | | | | |
| Sample 3- 50% | 38 | seconds | 42 | seconds | 37 | seconds | 80 | seconds |
| oxidized dextran | n.t. | | n.t. | | n.t. | | n.t. | |
| (12.5% w/v), | | | | | | | | |
| PAMAM dendrimer | | | | | | | | |
| (15% w/v), pH 9.5 | | | | | | | | |
| Sample 4- 75% | 27 | seconds | 32 | seconds | 34 | seconds | 45 | seconds |
| oxidized dextran | n.t. | | n.t. | | n.t. | | n.t. | |
| (12.5% w/v), | | | | | | | | |
| PAMAM dendrimer | | | | | | | | |
| (15% w/v), pH 9.5 | | | | | | | | |
| Sample 5- 50% | 62 | seconds | 70 | seconds | 76 | seconds | n.t. | |
| oxidized dextran | n.t. | | <1.5 | min | <2 | min | n.t. | |
| (12.5% w/v), | | | | | | | | |
| PAMAM dendrimer | | | | | | | | |
| (15% w/v), pH 9.2 | | | | | | | | |

Testing of the compositions of the foregoing table revealed that Sample 5 had a faster degradation time than Sample 1, as depicted at FIG. 4.

Based on the results of the initial screening, a number of hydrogels formulations were produced and tested, including those described in the following table:

| | Dextran | | Hydrogel Solid |
|---|---|---|---|
| | Formulation | Dendrimer | Content (by |
| Variable | (50% Oxidized) | Formulation | weight) |
| Dextran solid | 25% w/v | 15% w/v | 20% |
| content | dextran | dendrimer, | |
| | in PBS | pH 9.2 | |
| | 12.5% w/v | 15% w/v | 13.75% |
| | dextran | dendrimer, | |
| | in PBS | 5 ug/uL | |
| | | doxorubicin, | |
| | | pH 9.2 | |
| | 15% w/v | 15% w/v | 15% |
| | dextran | dendrimer, | |
| | in PBS | 5 ug/uL | |
| | | doxorubicin, | |
| | | pH 9.2 | |
| | 20% w/v | 15% w/v | 17.5% |
| | dextran | dendrimer, | |
| | in PBS | 5 ug/uL | |
| | | doxorubicin, | |
| | | pH 9.2 | |
| | 25% w/v | 15% w/v | 20% |
| | dextran | dendrimer, | |
| | in PBS | 5 ug/uL | |
| | | doxorubicin, | |
| | | pH 9.2 | |
| Drug formulation | 12.5% w/v | 15% w/v | 13.75% |
| polymer | dextran | dendrimer, | |
| | in PBS | pH 9.2 | |
| | 5 ug/uL | | |
| | doxorubicin | | |
| Overall solid | 20% w/v | 24% w/v | 17% |
| content | dextran | dendrimer, | |
| | in PBS | pH 9.2 | |
| | 7.5% w/v | 9% w/v | 8.25% |
| | dextran | dendrimer, | |
| | in PBS | 2 ug/uL | |
| | | doxorubicin, | |
| | | pH 9.2 | |
| | 12.5% w/v | 15% w/v | 13.75% |
| | dextran | dendrimer, | |
| | in PBS | 2 ug/uL | |
| | | doxorubicin, | |
| | | pH 9.2 | |
| | 15% w/v | 18% w/v | 16.5% |
| | dextran | dendrimer, | |
| | in PBS | 2 ug/uL | |
| | | doxorubicin, | |
| | | pH 9.2 | |
| | 20% w/v | 24% w/v | 22% |
| | dextran | dendrimer, | |
| | in PBS | 2 ug/uL | |
| | | doxorubicin, | |
| | | pH 9.2 | |

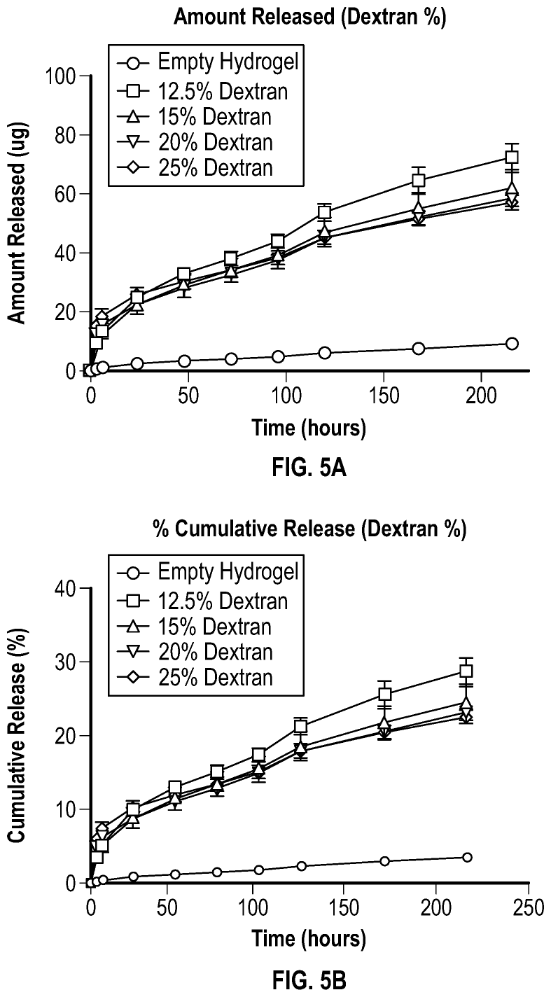

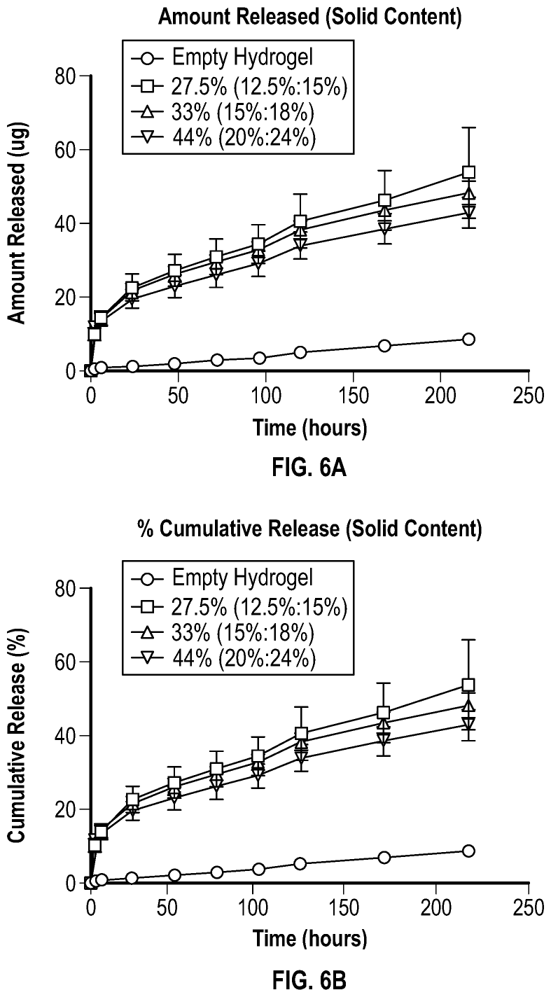

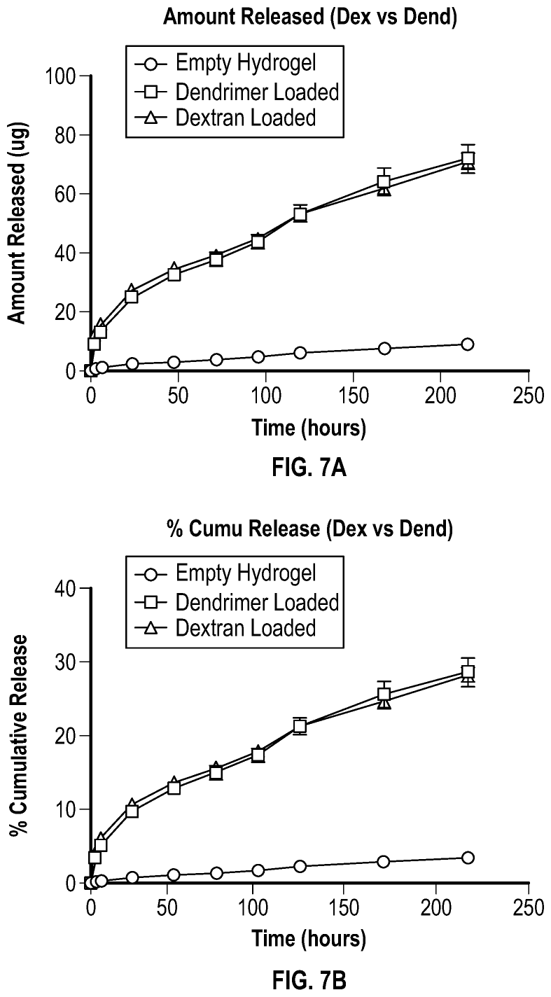

Doxorubin release profiles of the foregoing compositions were tested. As depicted at FIG. 5A and FIG. 5B, increasing the w/v % of dextran did not substantially impact the doxorubin release profiles.

The testing of this example also revealed that changing the solid content of the tested compositions did not substantially impact the doxorubicin release profiles, as depicted at FIG. 6A and FIG. 6B.

Further tests of this example also indicated that loading the doxorubicin with dextran or dendrimer did not substantially change the doxorubin release profiles, as depicted at FIG. 7A and FIG. 7B.

Changing the amount of doxorubin loaded into the foregoing compositions, however, slightly altered the doxorubicin release profiles, as depicted at FIG. 8A and FIG. 8B.

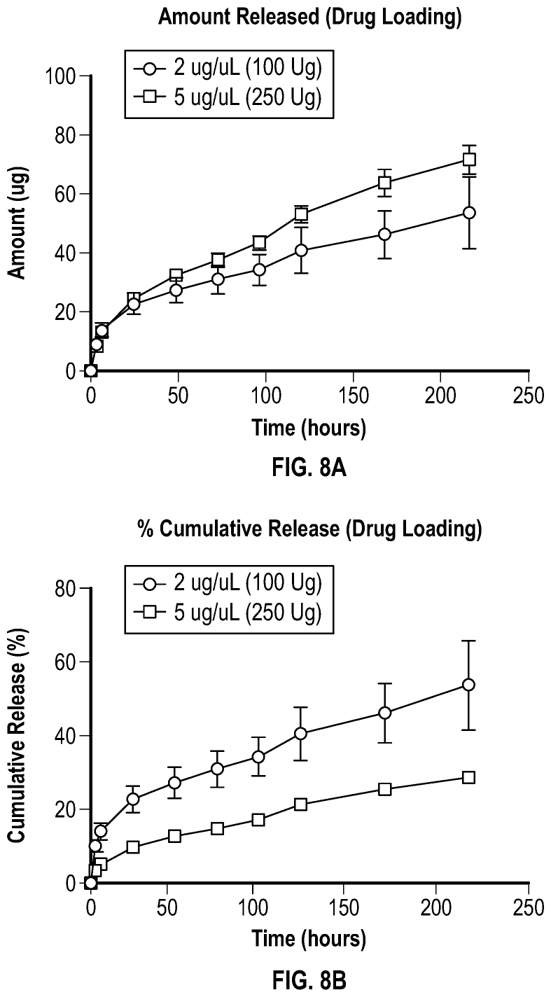

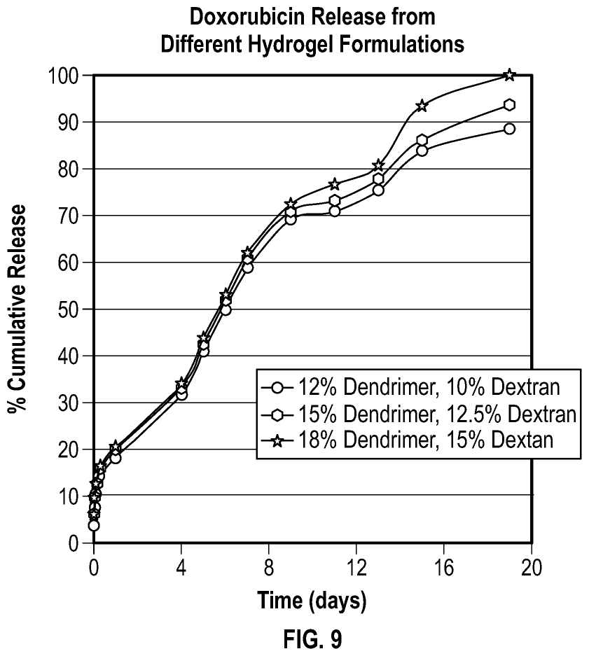

Each of the hydrogels tested in this example, however, provided sustained release of doxorubicin, as depicted at FIG. 9. After 19 days, the “12% dendrimer, 10% dextran” hydrogel had a cumulative release percentage of slightly less than 90%, the “15% dendrimer, 12.5% dextran” hydrogen had a cumulative release percentage of greater than 90%, and the “18% dendrimer, 15% dextran” had a cumulative release percentage of 100%.

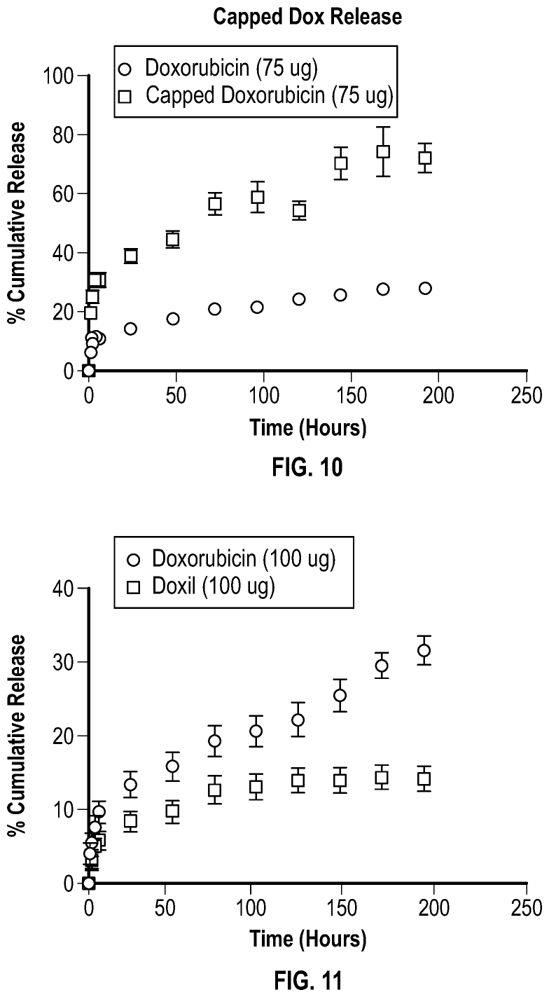

Also tested was the release of primary amine-capped doxorubicin. According to the testing, capped doxorubicin achieved a higher cumulative release percentage than doxorubicin, as depicted at FIG. 10. Also compared was the cumulative release percentage of doxil and doxorubicin. As depicted at FIG. 11, a greater cumulative percentage of doxorubicin was released over 200 hours.

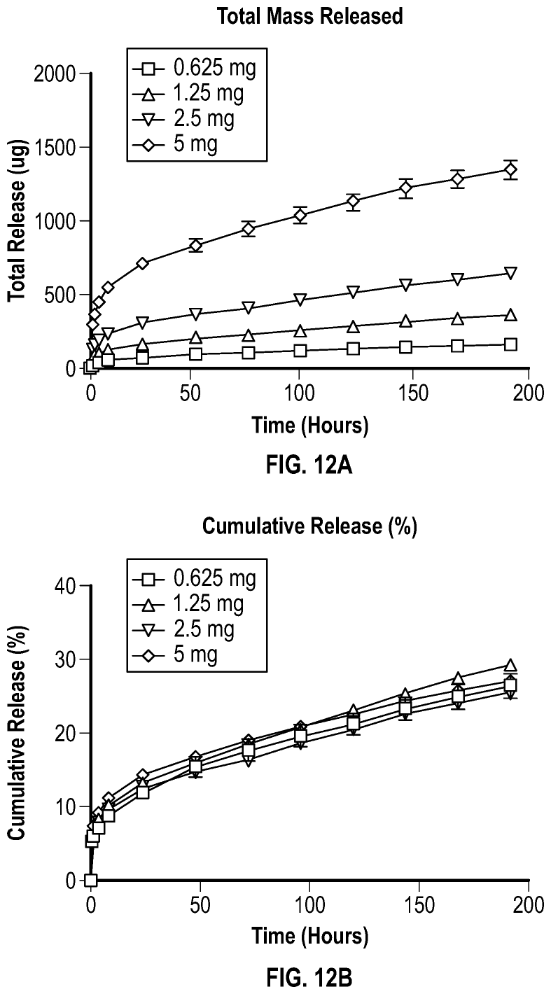

The in vitro release of acriflavine also was tested. An injectable hydrogel formulation (75% oxidized dextran, 12.5% w/v; PAMAM dendrimer, 15% w/v, pH 9.2) was prepared with four different loading concentrations (0.625 mg, 1.25 mg, 2.5 mg, and 5 mg, each of which had been previously injected locally in the brain). The cumulative release profiles were similar for the different loading amounts, as depicted at FIG. 12A and FIG. 12B. Acriflavine retained its activity upon release, as expected.

After hydrogels of this example were injected into the brains of mice, magnetic resonance imaging (MRI) was used to evaluate the persistence of the hydrogels. The hydrogels persisted for at least 20 days, according to MRI scans collected at injection, and days 2, 12, and 20 thereafter.

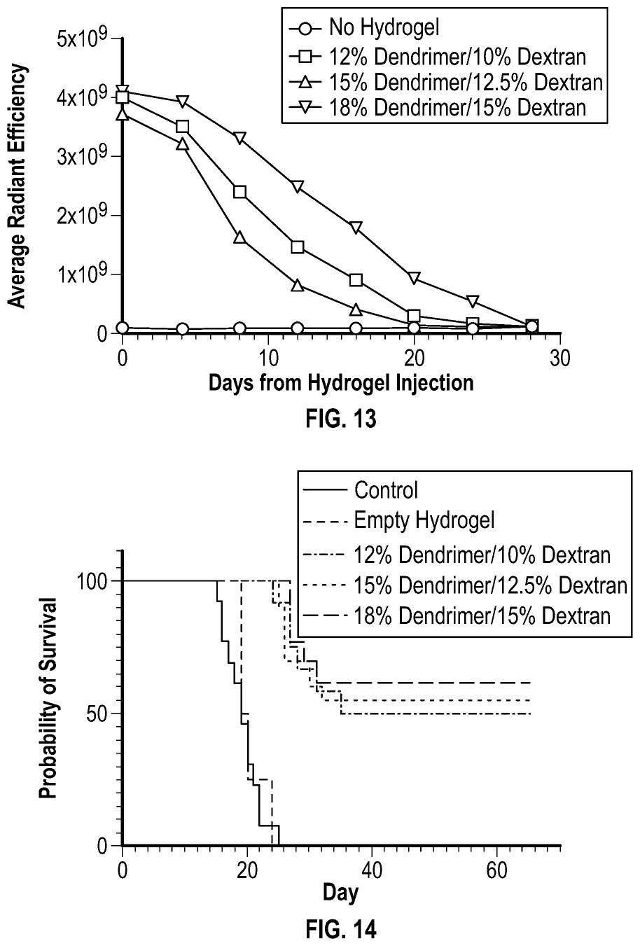

In addition to their persistence, the degradation of the hydrogels also was studied, specifically in vivo hydrogel degradation by IVIS for formulations having a different solids content. A plot of average radiant efficiency v. the number of days from hydrogel injection is depicted at FIG. 13.

A therapy retention and tissue penetration study was designed and conducted. The objective of this study was to assess the retention, cell uptake, and tissue penetration of doxorubicin when released from the hydrogel, and compare these to the intratumoral injection of the therapy. The following table describes the study:

| Day | Activity |
|---|---|
| 0 | GL261 Tumor Inoculation |
| 7 | IVIS Imaging for Tumor Size |
| 10 | Therapy Administration |
| 12 | Harvest, Section, Stain, and Image Brain Tissue |
| 15 | |

The results of this test indicated that the hydrogel delivery of doxorubicin improved the retention of doxorubicin compared to intratumoral injection. Increased doxorubicin tissue penetration over time also was observed when the doxorubicin was released from the hydrogel.

Further testing demonstrated that the solid content of the hydrogel formulations did not significantly impact the outcome of local dox therapy, as depicted at FIG. 14.

Another test was designed and conducted to evaluate whether sustained local delivery of doxorubicin using the hydrogels of this example could extend survival in orthotopic GL261 tumor-bearing mice. The following table describes the study, and these steps were applied to four groups (untreated control, empty hydrogel, intratumoral injection of doxorubicin, and hydrogel delivery of doxorubicin (N=6-10 per group; C57BL/6 mice)):

| Day(s) | Activity |
|---|---|
| 0 | GL261 Tumor Implantation |
| 7 | IVIS Imaging for Tumor Size |
| 10-14 | Administration of Treatment |
| 15-50 | Monitoring of Mouse Health, Survival, and Tumor Burden |

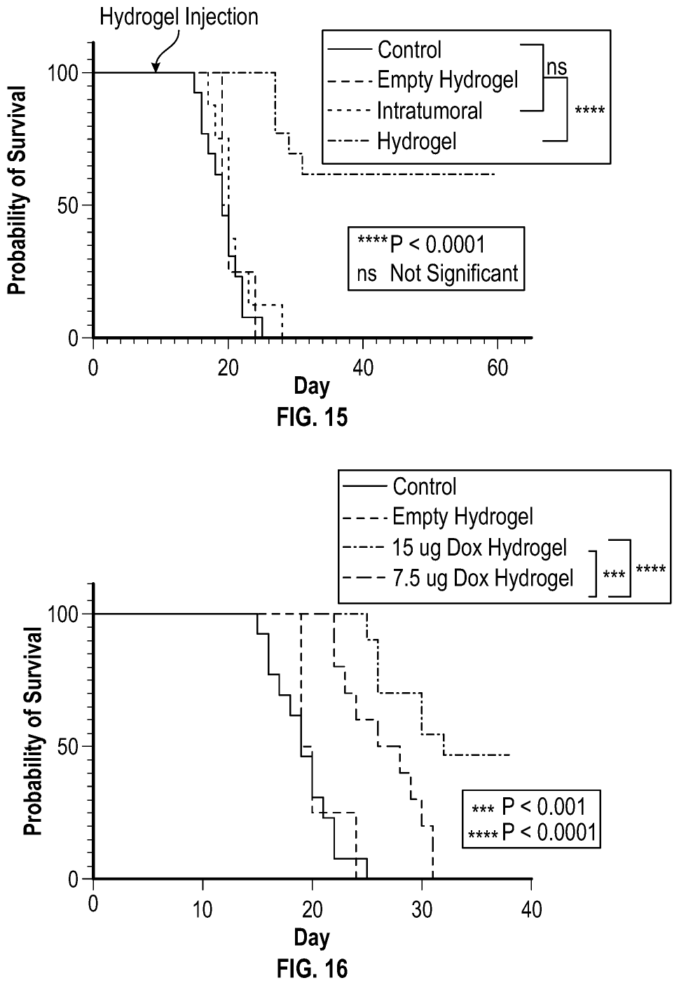

This test indicated that the local hydrogel delivery of doxorubicin significantly enhanced survival in orthotopic tumor-bearing mice compared to intratumoral (IT) injection, as depicted at FIG. 15. The results also indicated that the survival efficacy of the hydrogel delivery of doxorubicin was dose-dependent, as depicted at FIG. 16. FIG. 16 demonstrates that the probability of survival was similar for the control and empty hydrogel, and that the hydrogel containing 15 μg of doxorubicin resulted in a greater probability of survival than the hydrogel containing 7.5 μg of doxorubicin.

Another study was designed and tested to determine whether the local delivery of doxorubicin could enhance the efficacy of current oral temozolomide chemotherapy. The following table describes the study, which was applied to six groups (untreated control, empty hydrogel, oral temozolomide (TMZ), hydrogel delivery of doxorubicin, hydrogel delivery of doxorubicin plus oral TMZ) (N=6-10 per group, C57BL/6 mice)):

| Day(s) | Activity |
|---|---|
| 0 | GL261 Tumor Implantation |
| 7 | IVIS Imaging for Tumor Size |
| 10-14 | Administration of Treatment (Systemic Delivery of TMZ, daily |
| | on days 10-14; Hydrogel Delivery of Doxorubicin on Day 10) |
| 15-50 | Monitoring of Mouse Health, Survival, and Tumor Progression |

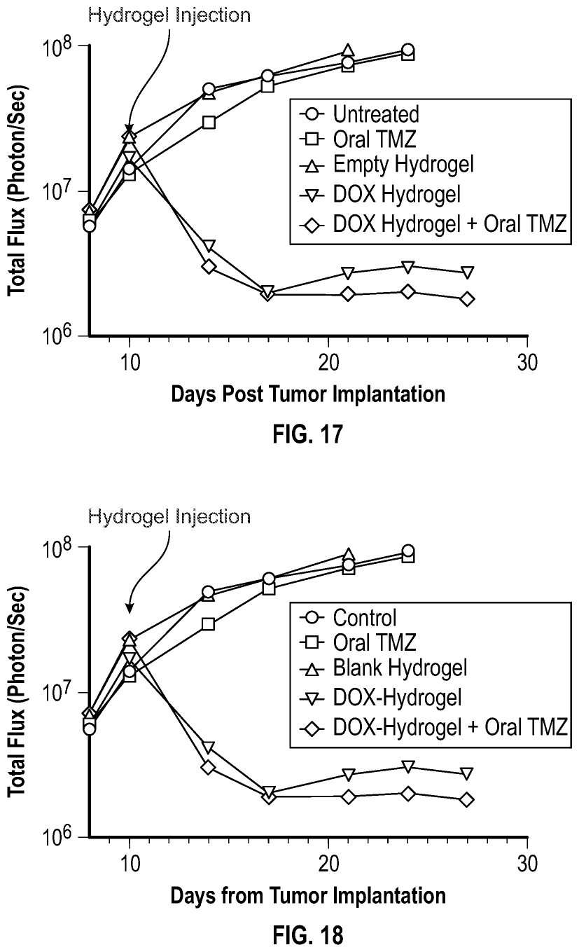

The results indicated that the local delivery of doxorubicin reduced tumor burden in mice bearing intracranial tumors, as depicted at FIG. 17. Tumor burden bioluminescence analysis was conducted throughout the test, and the data is depicted at FIG. 18.

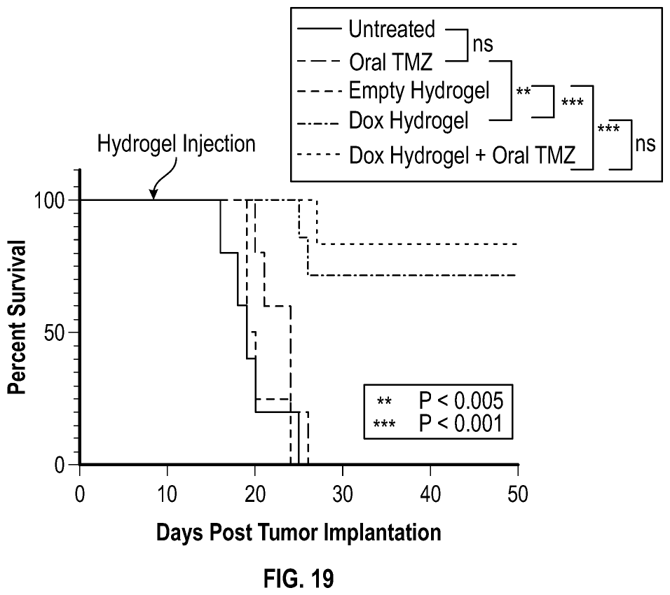

For the tests of this example, there was no significant benefit in median OS for mice receiving a combination oral TMZ and hydrogen doxorubicin therapy. This conclusion is based, at least in part, on the plot at FIG. 19.

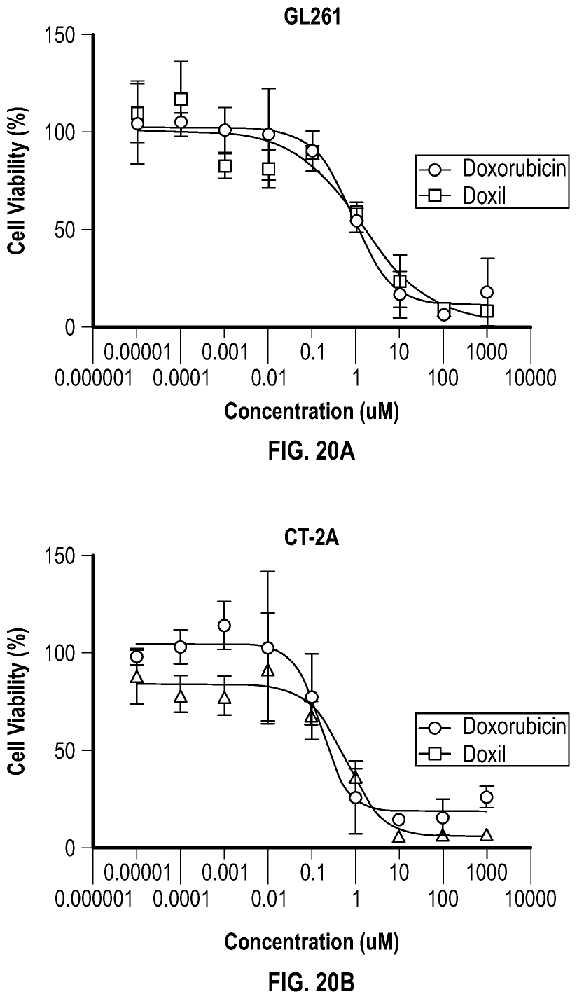

Comparative testing indicated that doxil had a potency comparable to doxorubicin in eliminated glioblastoma cells in vitro, as evidenced by FIG. 20A and FIG. 20B, which depict the cell viabilities of the following therapies:

| Therapy | GL261 | CT-2A |
|---|---|---|
| Doxorubicin | 0.83 μM | 0.17 μM |
| Doxil | 1.40 μM | 0.60 μM |

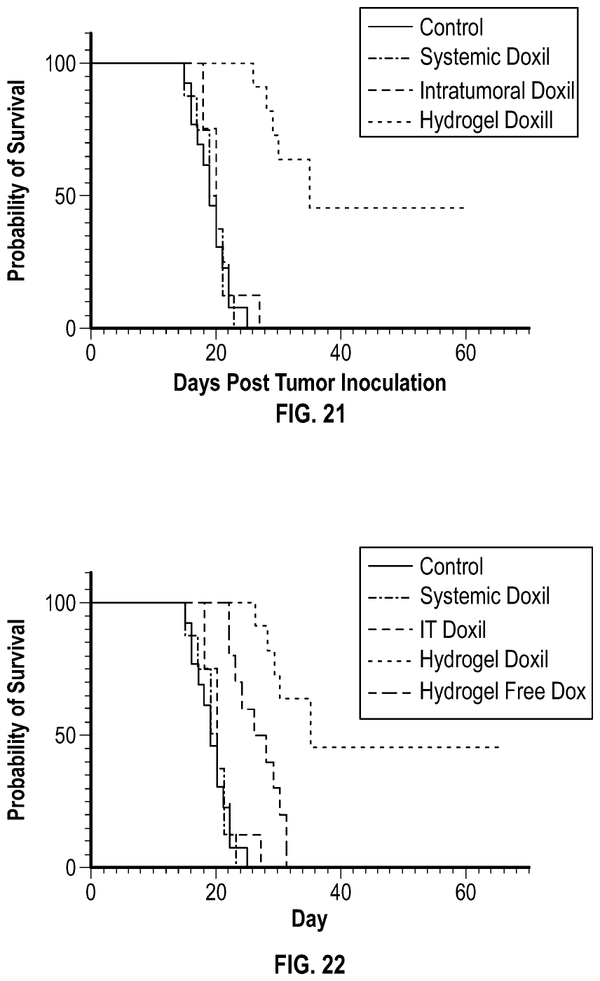

Further testing indicated that clinically-available nanoparticle DOX (DOXIL® chemotherapy) results in increased survival when delivered using the hydrogels of this example to tumor-bearing mice. This conclusion was based, at least in part, on the data depicted at FIG. 21. Clinically-available nanoparticle DOX (DOXIL® chemotherapy) was as effective as hydrogel free doxorubicin at half of the concentration (7.5 μg), as depicted at FIG. 22.

A study also was designed to test whether local doxorubicin delivered via hydrogel could eradicate patient-derived xenograft orthotopic tumors in nude mice. The study, which is described in the following table, was applied to six groups (untreated control, empty hydrogel, oral TMZ, hydrogel delivery of doxorubicin (7.5 μg), hydrogel delivery of doxorubicin (15 μg), and hydrogel delivery of DOXIL® chemotherapy (7.5 μg) (N=6-10 per group, nude mice) (PDX characteristics: MGMT methylated, EGFR non-amplified, TP53 non-mutated).

| Day(s) | Activity |
|---|---|
| 0 | PDX Tumor Implantation |
| 7 | IVIS Imaging for Tumor Size |
| 10-14 | Administration of Treatment (Systemic Delivery of TMZ, daily |
| | on days 10-14; Hydrogel Delivery of Doxorubicin/DOXIL ® |
| | chemotherapy on Day 10) |
| 15-50 | Monitoring of Mouse Health, Survival, and Tumor Progression |

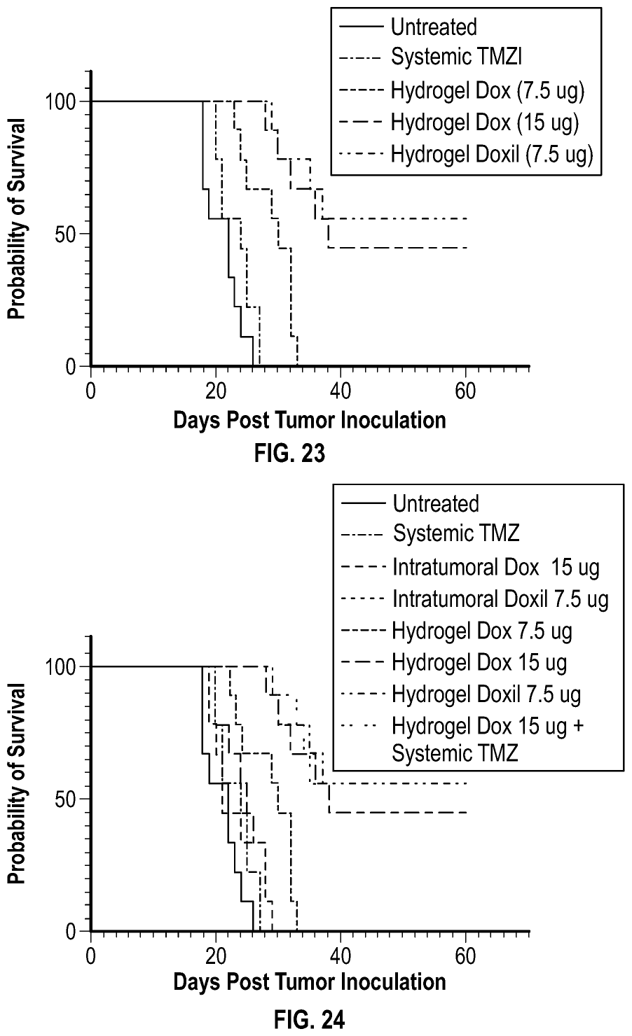

The results of the study, as depicted at FIG. 23, indicated that survival was significantly extended in orthotopic PDX tumor-bearing mice treated with hydrogel doxorubicin therapies, as seen in syngeneic models. At 60 days after tumor inoculation, the hydrogel delivery of DOXIL® chemotherapy (7.5 μg) resulted in a probability of survival of greater than 50%, and the hydrogel delivery of doxorubicin (15 μg) had a probability of survival slightly less than 50%.

Survival was significantly extended in orthotopic PDX tumor-bearing NSG mice treated with the various hydrogel doxorubicin therapies of this example. These results are depicted at FIG. 24.

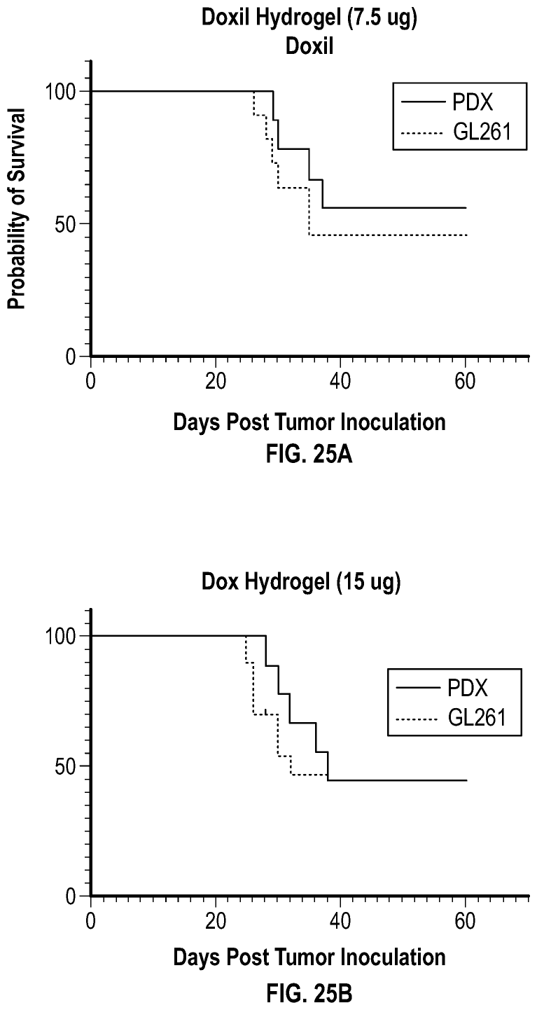

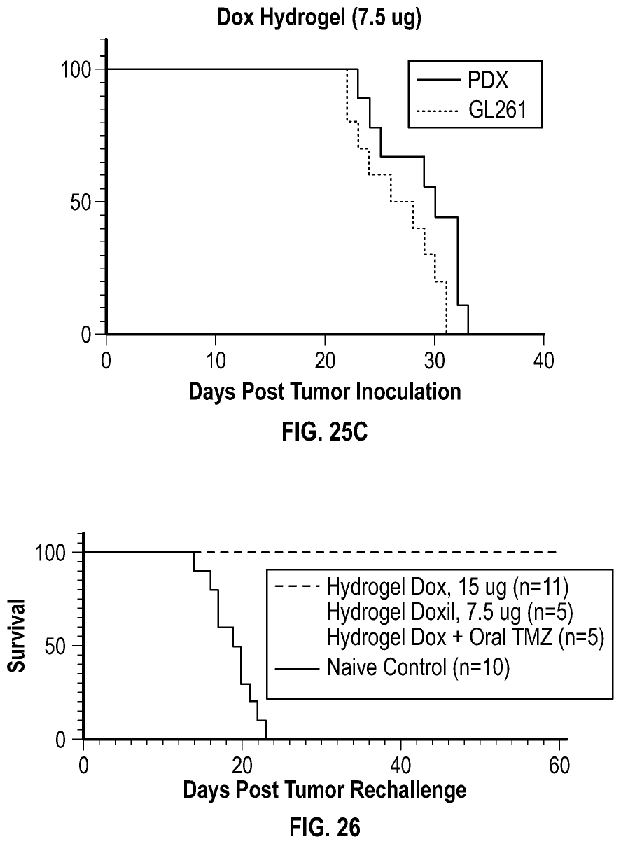

The testing of this example also indicated that there was no significant difference between PDX nude mice and GL261 C57BL6 mice survival (see FIG. 25A (DOXIL® chemotherapy hydrogel) (7.5 μg), FIG. 25B (doxorubicin hydrogel) (15 μg), FIG. 25C (doxorubicin hydrogel) (7.5 μg)). Insignificant differences obtained between survival curves for nude and C57BL6 mice for all therapies, but it should be noted that different cells were used. These results suggested that adaptive immune response may not play a significant role in eliminated tumor on primary challenge.

Another test was conducted to evaluate whether local doxorubicin therapy conferred protection to long-term surviving mice against tumor rechallenge. This study following the following parameters:

| Day | Activity |
|---|---|
| 0 | Primary Challenge (left hemisphere of brain) |
| 10-13 | Treatment |
| 70 | Tumor Rechallenge (right hemisphere of brain) |

All long-term surviving syngeneic mice treated with local hydrogel therapies rejected tumors upon contralateral hemisphere rechallenge. This conclusion was based on the data depicted at FIG. 26.

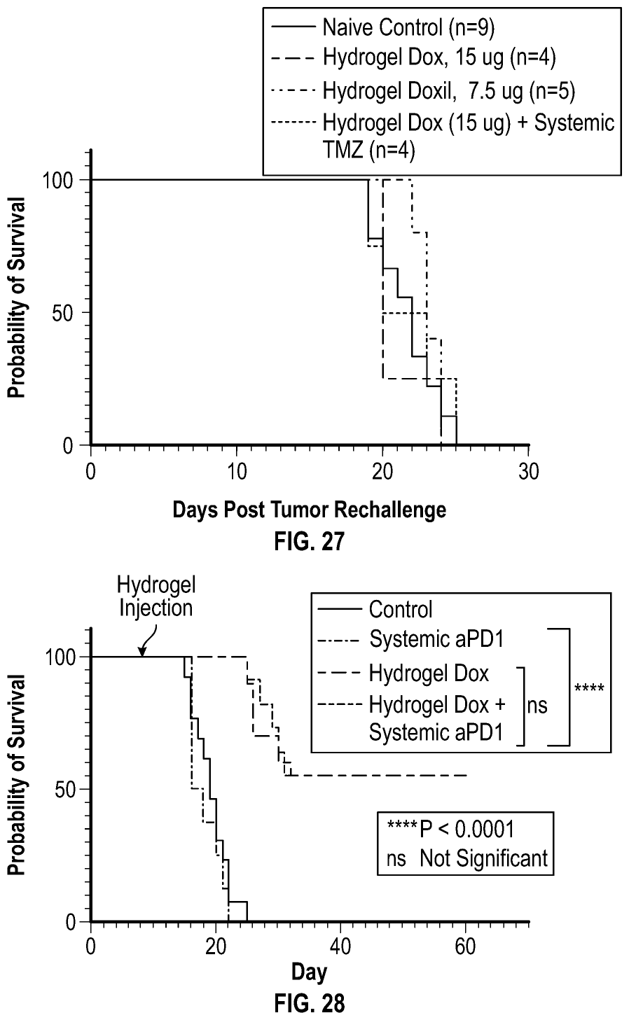

Also, long-term surviving PDX nude mice did not reject tumors upon contralateral hemisphere rechallenge, as depicted at FIG. 27.

Yet another study was conducted to determine whether local hydrogel doxorubicin chemotherapy can synergize with systemic aPD1 checkpoint blockade therapy. The dose for this study was 200 μg aPD1 given intraperitoneally, and four groups were tested (untreated, systemic aPD1, intracranial doxorubicin hydrogel, intracranial doxorubicin hydrogel plus systemic aPD1) (C57BL/6 mice, N=6-10 per group):

| Day | Activity |
|---|---|
| 0 | Primary Challenge (1.3 × 10⁵ GL261) |
| 10 | Doxorubicin hydrogel injection/aPD1 injection |
| 12 | aPD1 injection |
| 14 | aPD1 injection |
| 60 | Track Tumor Growth/Track Body Weight |

The combination of local doxorubicin therapy with systemic aPD1 did not enhance survival of glioblastoma GL261-bearing mice, as depicted at FIG. 28. Not wishing to be bound by any particular theory, it was believed that one or more factors could be driving the lack of synergism between local doxorubicin chemotherapy and aPD1. For example, regarding the drug release and biodistribution profiles of the therapies, doxorubicin may reduce infiltrating T cells, and/or systemic aPD1 may not reach target tissues with necessary pharmacokinetic profiles. Regarding the immunosuppressive tumor microenvironment (TME), the factors may include limited T cell infiltration and penetration into TME with chemotherapy, and/or the chemotherapy is unable to eliminate or revert immunosuppressive macrophages.

Primary tumor immunophenotyping-Five mice were tested for each treatment group (untreated, doxorubicin hydrogel), and the mice were sacrificed 14 days after tumor inoculation (4 days after therapy). The tissues tested included tumor, spleen, bone marrow, blood, and tumor draining lymph nodes. The panels for this test included memory T cells, macrophages, and dendritic cells/MDSCs.

| Day | Activity |
|---|---|
| 0 | GL261 or CT-2A Orthotopic Tumor Injection |
| 7 | IVIS Imaging for Tumor Size |
| 10 | Treatment Administration |
| 10-14 | Monitor Mouse Weight, Health, and Tumor Progression |
| 14 | Harvest Organs for Immunophenotyping (tumor, draining lymph |
| | nodes, spleen, bone marrow, and blood)(flow cytometry, |
| | immunohistochemistry, and ex vivo assays) |

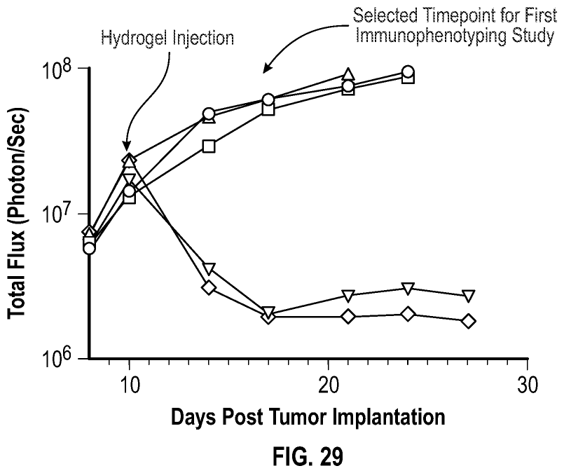

The majority of untreated tumors did not have significant numbers of immune cells for T cell panel. CD45+ and CD3+ subsets of live cells were <1000 events in ⅗ untreated tumors. Tested were different enrichment steps to increase the number of immune cells in samples (magnetic beads, different Percoll gradients). A plot of total flux (photon/sec) v. days post tumor implantation is depicted at FIG. 29.

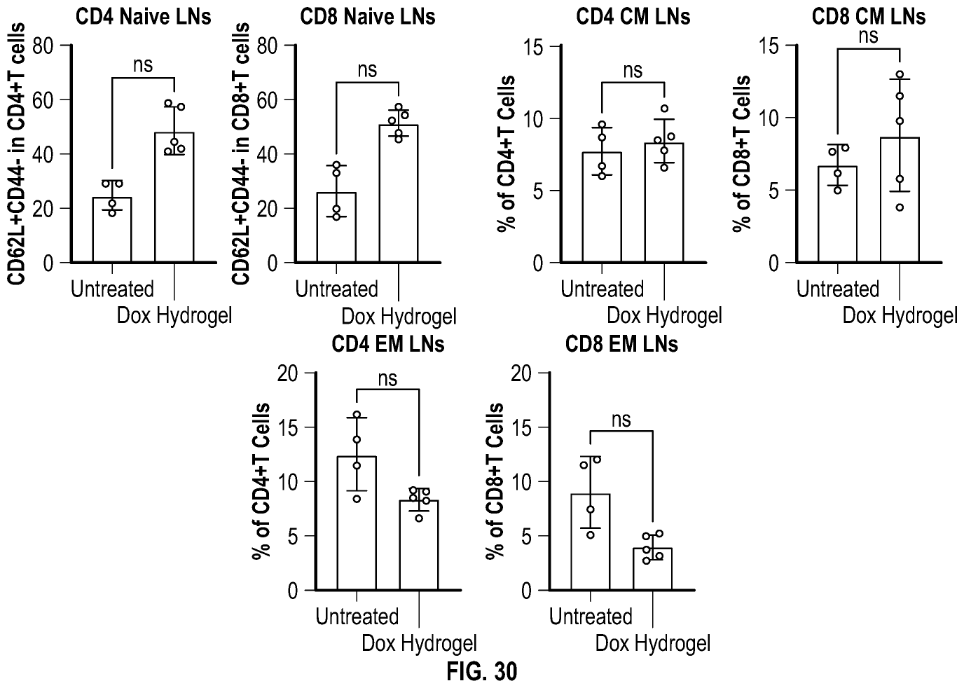

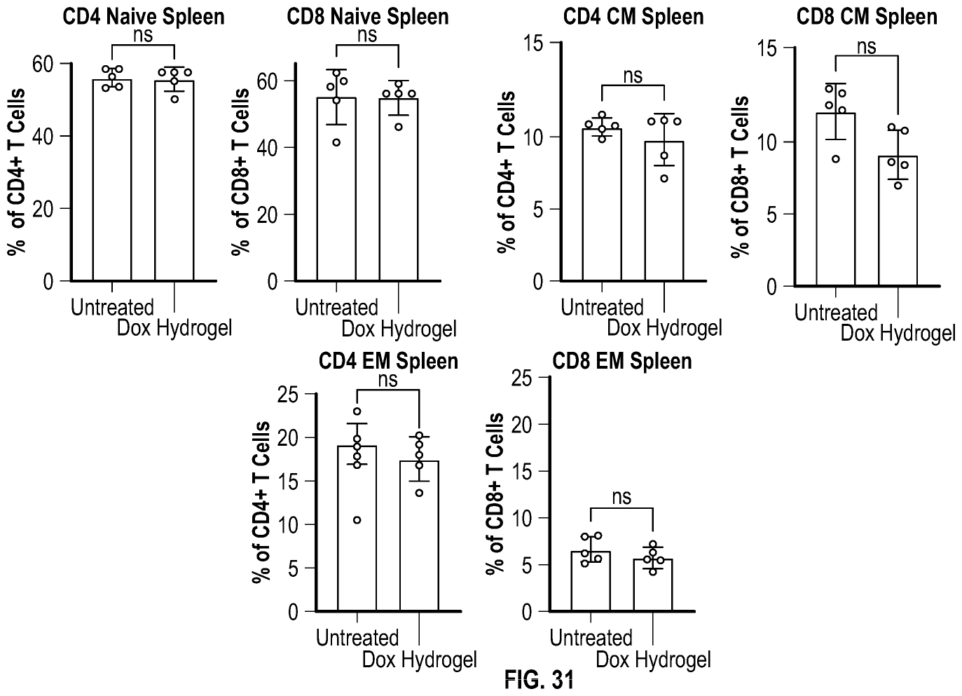

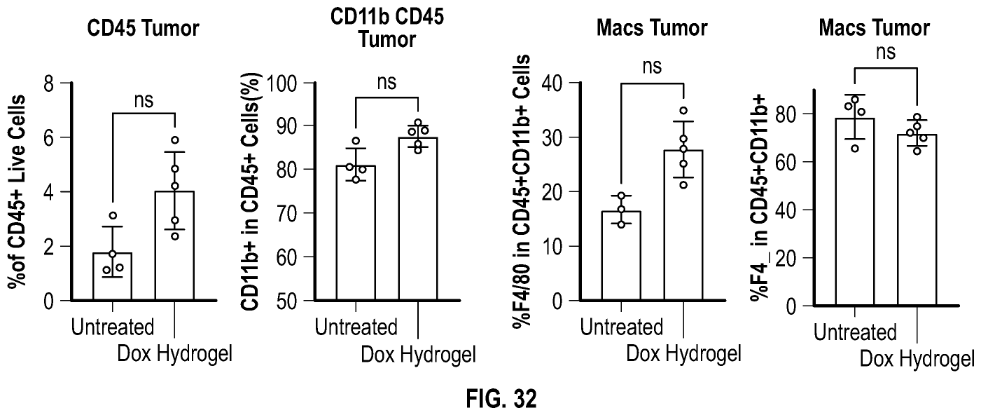

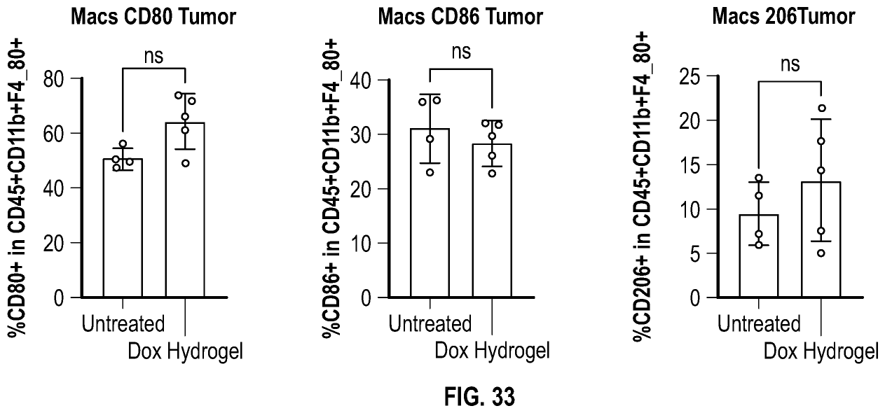

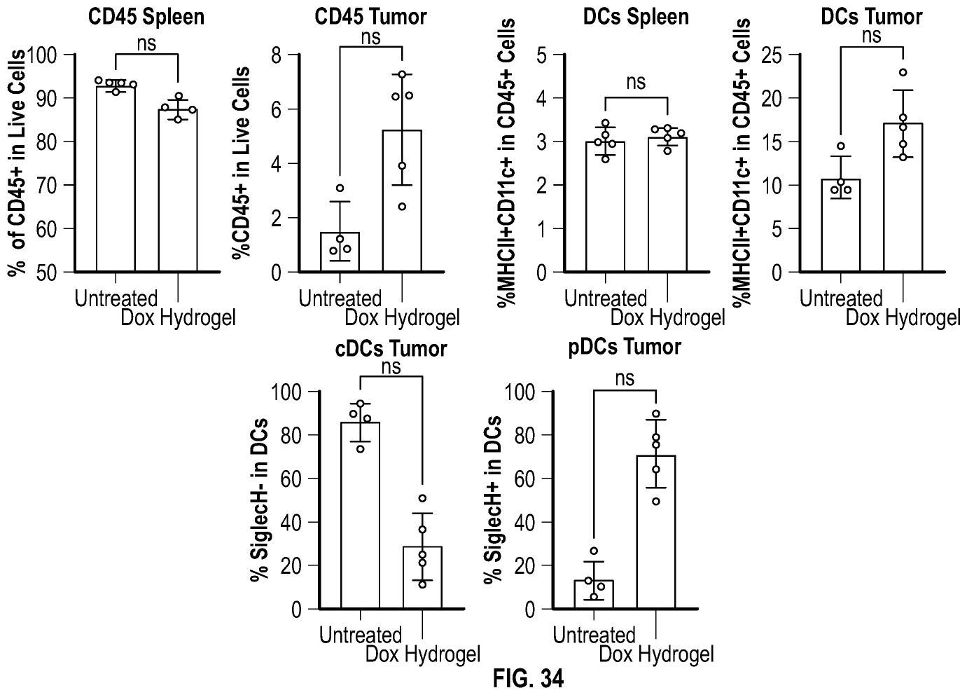

The increase in naïve T cells in the lymph nodes four days after therapy compared to untreated mice are depicted at FIG. 30. No other significant differences in T cells were noted in the tumor draining lymph nodes. Only a slight decrease in central memory CD8 T cells in the spleen were observed, as depicted at FIG. 31. The only other difference in spleen cell immune infiltrate was a slight decrease in CD45+ cells in the treated groups. Macrophages contributed to the increase in CD45+ cells in the tumor upon doxorubicin hydrogel treatment, as depicted at FIG. 32. A slight increase in M1 polarization marker CD80+ in doxorubicin treated tumors was observed, at depicted at FIG. 33. A preliminary analysis suggested significant shifts in dendritic cells populations, as depicted at FIG. 34. It should be noted, however, that low numbers of DCs in the untreated tumors (˜1000s) complicated the analysis of the cDC/pDC populations.

### Example 2-Further Testing and Analysis

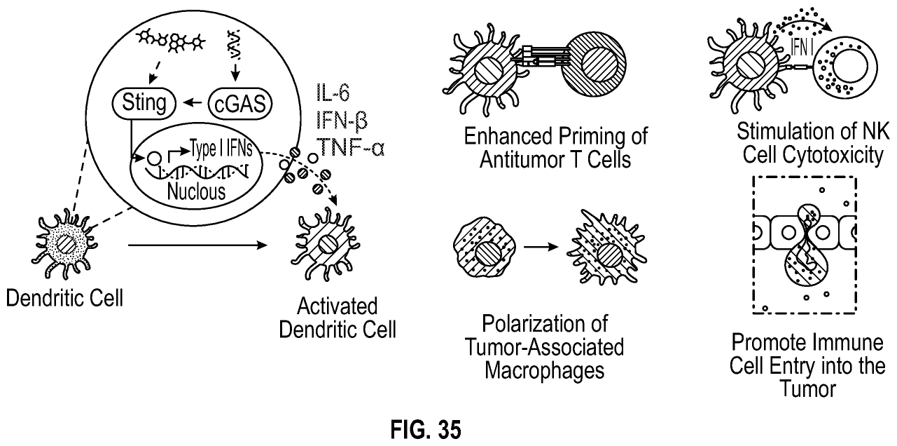

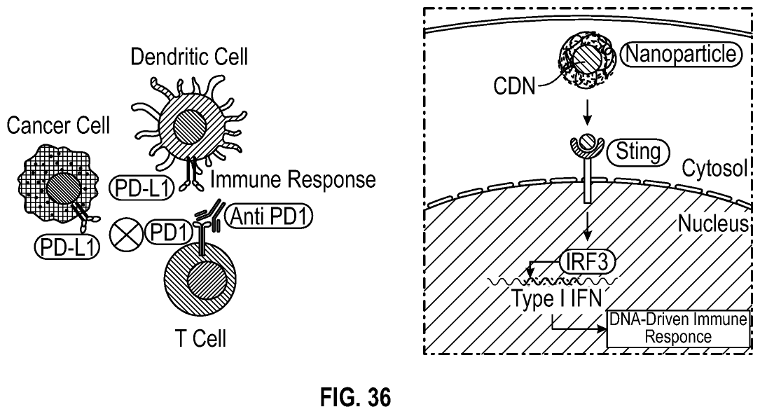

STING (cGAS/stimulator of interferon genesagonists) were understood to drive anticancer immune activity through the production of type I interferons and other proinflammatory cytokines (FIG. 35). Nanoparticles may be used in the cGAS-STING pathway. Cyclic dinucleotides (CDNs) may be potential cancer immunotherapy drugs, and nanoparticle mediated delivery may be used (FIG. 36).

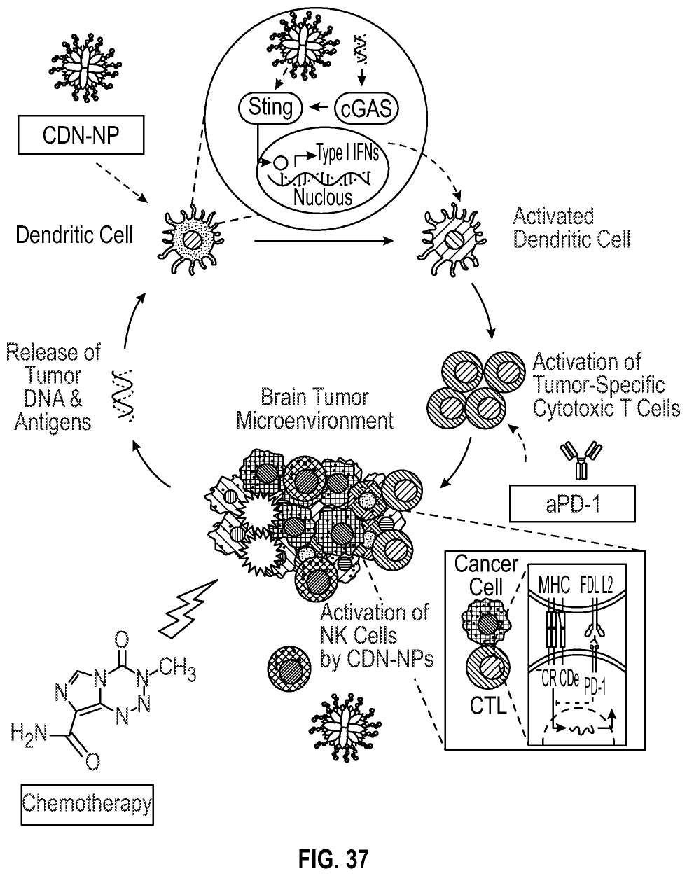

It is hypothesized that local hydrogel delivery of combination chemoimmunotherapy may (1) modulate key components of the tumor microenvironment, (2) enhance antitumor immune priming, and/or (3) increase the efficacy of checkpoint blockade therapy (FIG. 37).

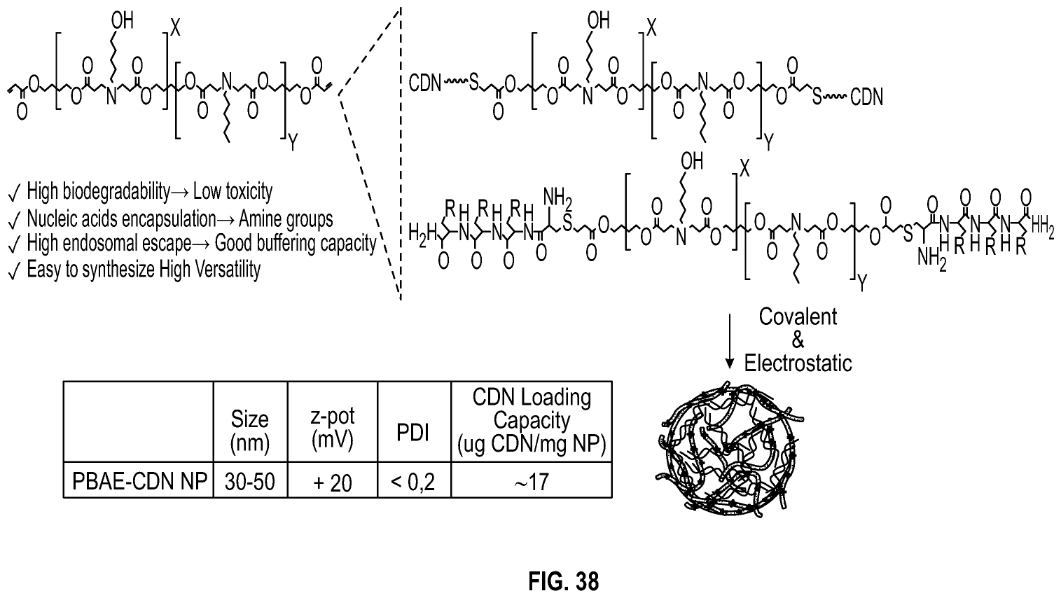

A nanoparticle-based STING agonist formulation may have or facilitate high biodegradability, low toxicity, nucleic acids encapsulation (amine groups), high endosomal escape (good buffering capacity), easy synthesis (high versatility) (FIG. 38).

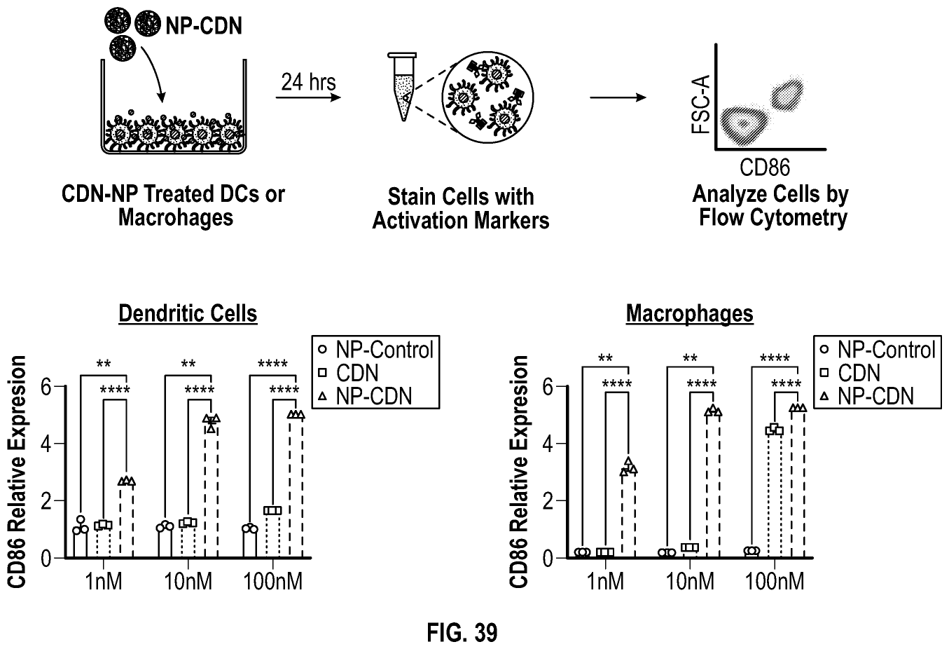

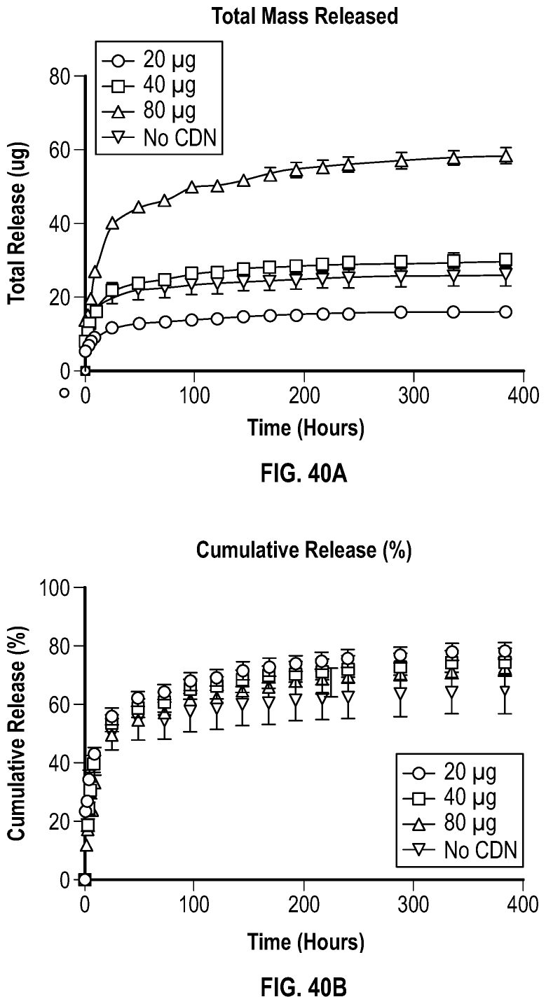

CDN nanoparticles stimulated dendritic cells and macrophages, as depicted at FIG. 39. The hydrogel compositions of the examples mediated release of the CDN nanoparticles in vitro, as depicted at FIG. 40A and FIG. 40B.

The high hydrophilicity and negative charge of CDNs can hinder their delivery into cells and thus their clinical potential, limiting their delivery the tumor to intratumoral injection. While CDNs are currently undergoing clinical trials, there are concerns that simple intratumoral CDN injection is a suboptimal means to stimulate the cytosolic STING signaling pathway. This example, however, describes polymer-based, CDN-conjugated, nanoparticles to permit systemic delivery of CDN, that otherwise clears from circulation within minutes, that is programmed to be released in the cell cytosol and its safety, efficacy and mechanism of action in multiple mouse tumor models have been studied.

These NPs were made of PBAEs that were modified with arginine residues enhancing the biocompatibility and endosomal escape ability of the NPs and complexed with CDN-conjugated PBAE chains with a cathepsin-sensitive bond enabling the release of CDN in the cell cytoplasm.

A local immunotherapy MTD pilot study was designed and conducted. The parameters of the study appear at the following tables. GL261-luc tumor bearing mice were tested, and each group included 3 mice. The mice received 10 μL hydrogel injections, and IVIS imaging was used to evaluate luciferase-tumor burden, AF568-hydrogel degradation, and AF647-therapy release.

| Day | Activity |
|---|---|
| 0 | Tumor Implantation |
| 7 | Hydrogel Injection |
| After Day 7 | IVIS Imaging - Monitor Weight, Tumor Growth, Survival, |
| | Hydrogel Degradation, and Therapy Release |

| Therapy | Dose | |
|---|---|---|
| Empty Hydrogel Control | N/A | |
| | 60 | μg |
| aPD-1 Local Hydrogel | 150 | μg |
| | 300 | μg |
| | 60 | μg |
| aPD-L1 Local Hydrogel | 150 | μg |
| | 300 | μg |
| | 20 | μg |
| CDN-NPs* Local Hydrogel | 40 | μg |
| dendrimer + NHS-PEG-SH | 80 | μg |

*CDN-NPs = PAMAM dendrimer + NHS-PEG-SH linker + Aduro CDN-Mal (~30 CDN/dendrimer)

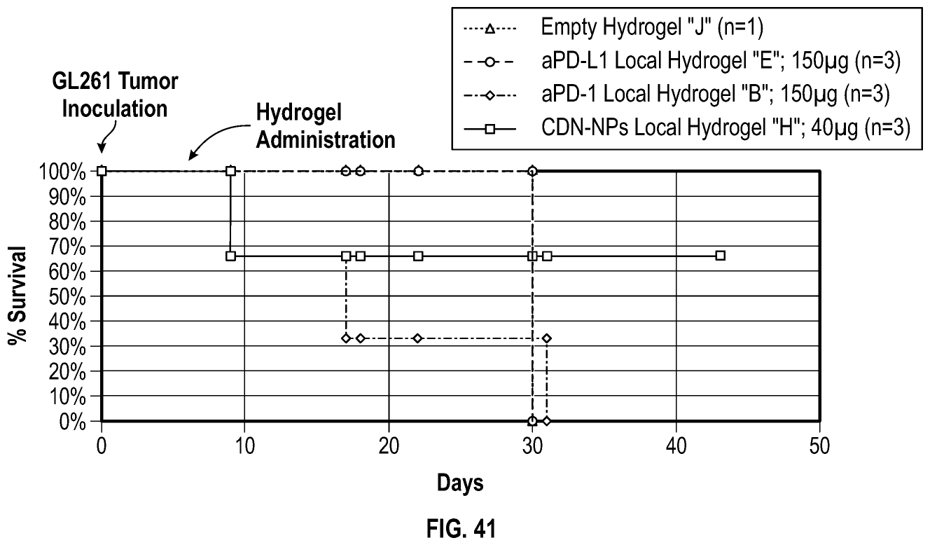

Results of the local immunotherapy pilot study are depicted at FIG. 41. The tumor burden imaging based on IVIS bioluminescence (Day 18) was collected for the empty hydrogel, aPD-1 hydrogel (150 micrograms), aPD-L1 hydrogel (150 micrograms), and CDN dendrimer NP hydrogel (40 micrograms CDN).

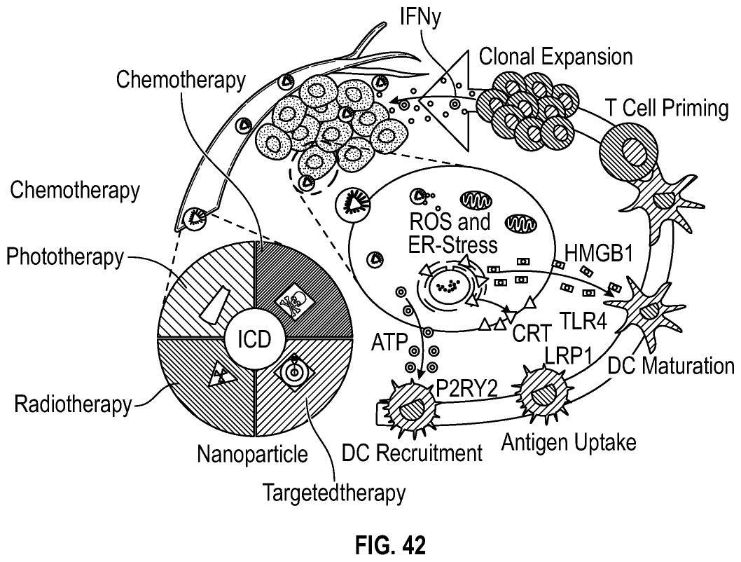

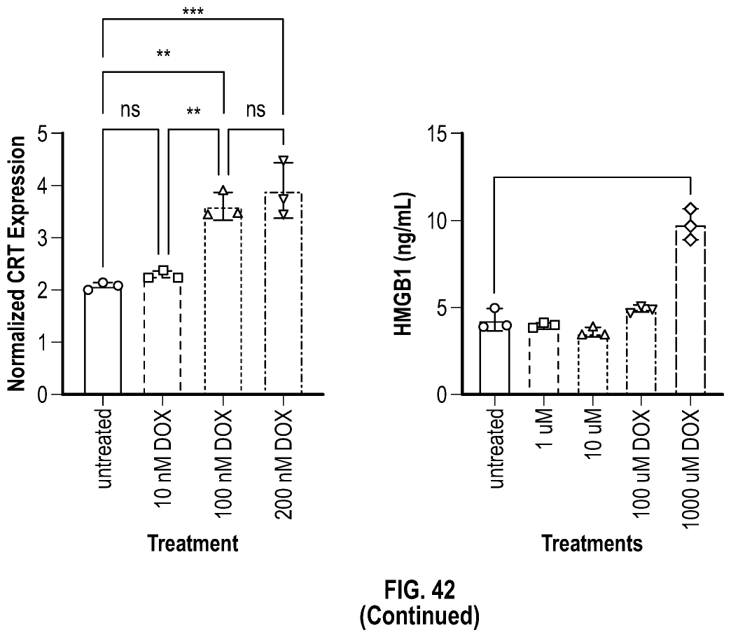

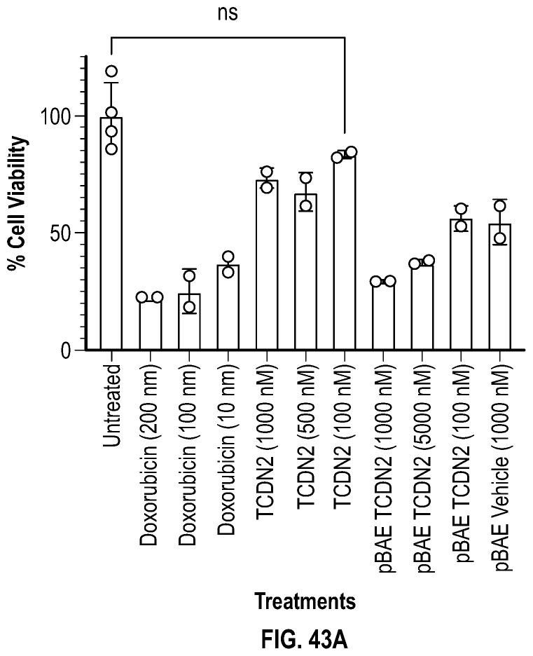

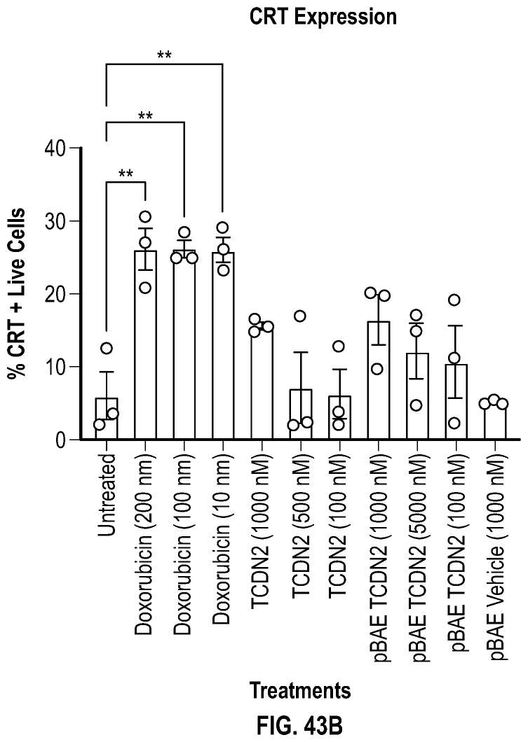

It was determined that doxorubicin treatment increase the surface expression of calreticulin on GBM cells, but did not impact HMGB1 excretion at relevant concentrations. Results are depicted at FIG. 42. Doxorubicin induced calreticulin (CRT) exposure pointing at immunogenic-cell death, as depicted at FIG. 43.

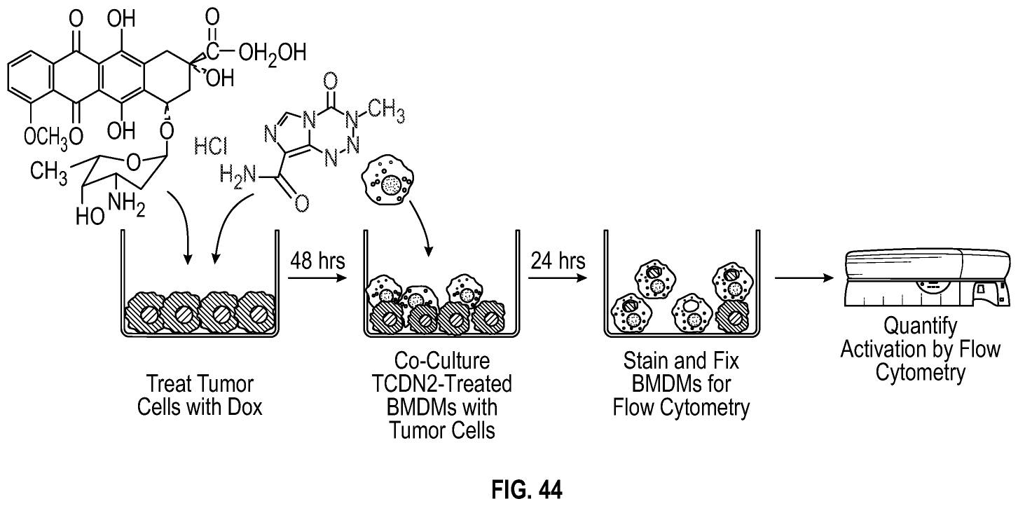

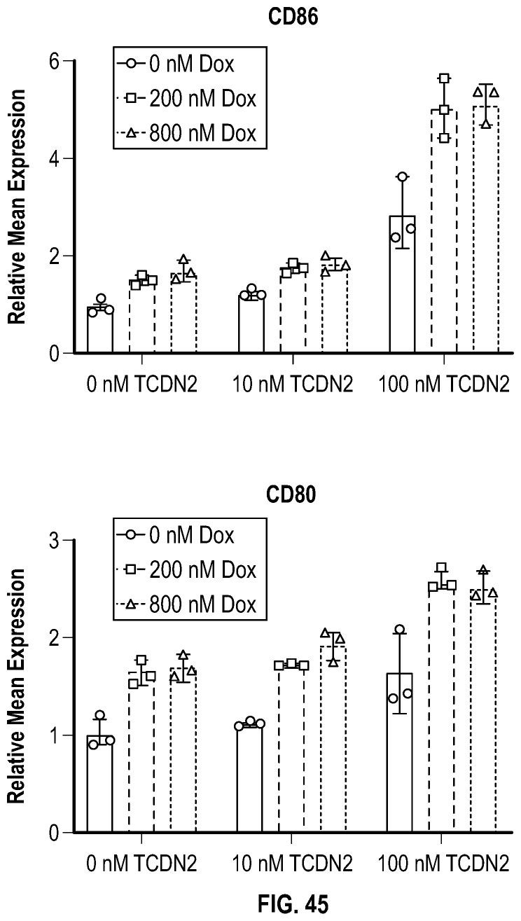

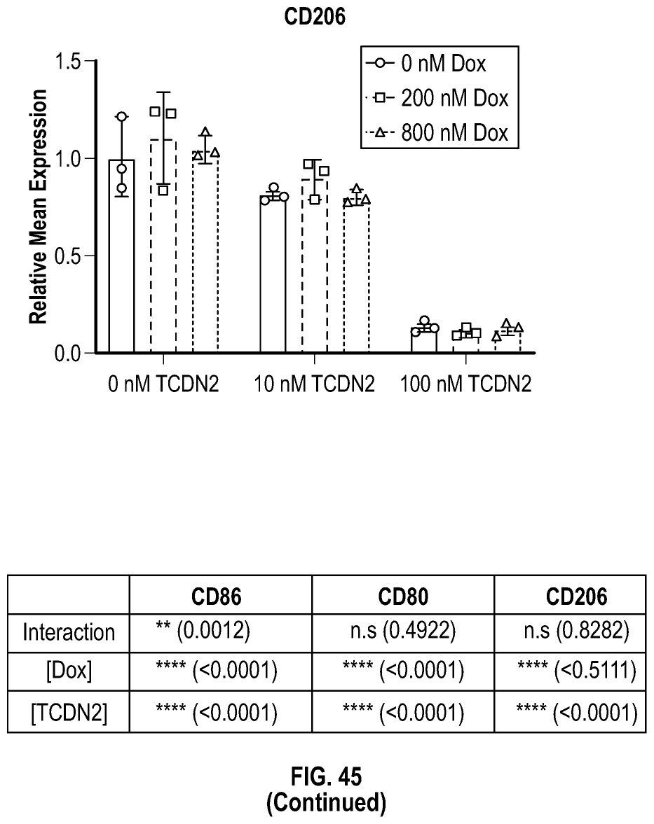

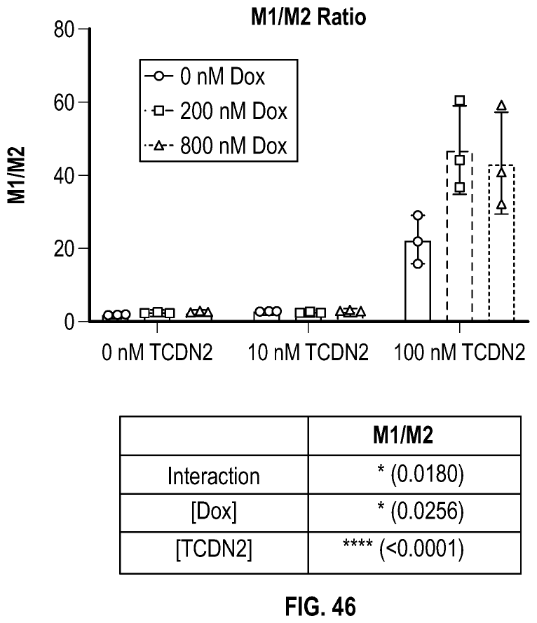

Also tested was whether chemoimmunotherapy can synergize to enhance bone-marrow derived macrophage activation (FIG. 44). As depicted at FIG. 45, BMDM CD 86 activation marker expression was enhanced by chemoimmunotherapy treatment. As depicted at FIG. 46, combination therapy increased the ratio of pro-inflammatory to anti-inflammatory BMDMs.

It was determined that the hydrogels of the foregoing examples, including adhesive hydrogels, effectively delivered therapy locally to treat malignant brain tumors in mice. For example, the hydrogel delivery of free or nanoparticle doxorubicin extended survival of mice bearing orthotopic syngeneic tumors of PDX tumors. Also, 100% of long-term surviving mice treated with local hydrogel doxorubicin therapies rejected tumors upon rechallenge. Hydrogel delivery of CDN nanoparticles is another strategy to treat GBM alone and in combination with doxorubicin chemotherapy.

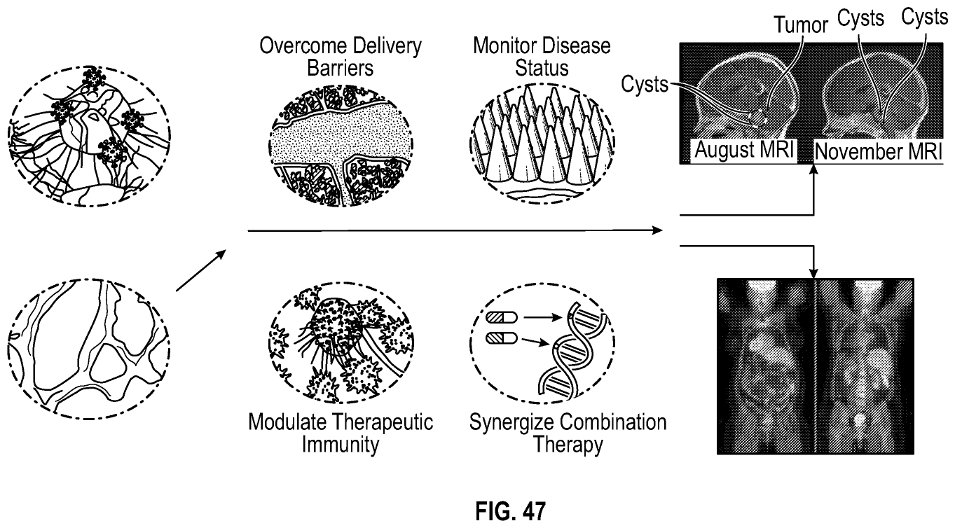

The compositions and methods described herein can allow for the engineering of immunity using materials to enhance therapeutic efficacy (FIG. 47).

1. A drug delivery composition comprising:
a hydrogel, wherein the hydrogel is an adhesive hydrogel; and
a drug dispersed in the hydrogel,
wherein the drug comprises a chemotherapy drug, an immunotherapy drug, or a combination thereof.

2. The drug delivery composition of claim 1, wherein—
the drug comprises the chemotherapy drug, and the chemotherapy drug comprises doxorubicin; and
the hydrogel comprises (i) a polymer component, wherein the polymer component comprises a polymer having three or more aldehyde groups, and (ii) a dendrimer component, wherein the dendrimer component comprises a dendrimer having at least 2 branches with one or more surface groups.

3. The drug delivery composition of claim 1, wherein the hydrogel comprises:
a polymer component, wherein the polymer component comprises a polymer having three or more aldehyde groups; and
a dendrimer component, wherein the dendrimer component comprises a dendrimer having at least 2 branches with one or more surface groups.

4. The drug delivery composition of claim 3, wherein 100% of the one or more surface groups comprise at least one primary or secondary amine.

5. The drug delivery composition of claim 3, wherein less than 75% of the one or more surface groups comprise at least one primary or secondary amine.

6. The drug delivery composition of claim 1, wherein the dendrimer is a generation 5 polyamidoamine (G5 PAMAM) dendrimer.

7. The drug delivery composition of claim 1, wherein the polymer comprises dextran.

8. The drug delivery composition of claim 1, wherein the chemotherapy drug comprises doxorubicin, and the doxorubicin is encapsulated in a liquid sphere.

9. The drug delivery composition of claim 8, wherein the liquid sphere is a liquid nanosphere.

10. The drug delivery composition of claim 1, wherein the immunotherapy drug comprises a cyclic dinucleotide.

11. The drug delivery composition of claim 10, wherein the cyclic dinucleotide is encapsulated in a nanoparticle, wherein the nanoparticle—
(i) comprises a poly-beta-amino-ester to which the cyclic dinucleotide is conjugated via a cathepsin-sensitive bond, wherein the poly-beta-amino-ester is optionally modified with arginine;
(ii) has an average diameter of about 30 nm to about 70 nm; or
(iii) a combination thereof.

12. The drug delivery composition of claim 1, wherein the drug delivery composition has a solid content of about 8% to about 25%, by weight.

13. The drug delivery composition of claim 1, wherein the hydrogel comprises phosphate buffered saline (PBS).

14. The drug delivery composition of claim 1, wherein the drug is present in the drug delivery composition at (i) a total amount of about 50 μg to about 500 μg, (ii) a total concentration of about 1 μg/μL to about 10 μg/μL, or (iii) a combination thereof.

15. The drug delivery composition of claim 3, wherein the three or more aldehyde groups of the polymer reversibly react with one or more amines of (i) the drug, (ii) the dendrimer, and/or (iii) one or more biological tissues to form imine bonds, and wherein the forming of the imine bonds and hydrolysis of the imine bonds controls, or contributes to, release kinetics of the drug from the hydrogel.

16. A method of treating a patient, the method comprising:
locally delivering the drug delivery composition of claim 1 to an intracranial region of the patient.

17-19. (canceled)

20. The method of claim 16, further comprising administering a second drug or therapy to the patient before, during, and/or after the locally delivering of the drug delivery composition.

21-24. (canceled)

25. The method of claim 16, wherein the drug is released from the drug delivery composition continuously for at least 24 hours.

26. The method of claim 16, wherein a cumulative percentage of the drug released from the drug delivery composition is at least 80%, by weight, of the drug within 10 days or less after the locally delivering of the drug delivery composition.

27. A kit for making a drug delivery composition, the kit comprising:
a first part which includes a first solution comprising a polymer component, wherein the polymer component comprises a polymer; and
a second part which includes a second solution comprising a dendrimer component, wherein the dendrimer component comprises a dendrimer having at least 2 branches with one or more surface groups;
wherein at least one drug is disposed in the first solution, the second solution, or both the first solution and the second solution, and
wherein the drug comprises a chemotherapy drug, an immunotherapy drug, or a combination thereof.

28-32. (canceled)
